(19) United States
(12) Patent Application Publication
(10) Pub. No.: US 2026/0077058 A1
(43) Pub. Date: Mar. 19, 2026

(54) PEPTIDE-TYPE NUCLEIC ACID CARRIER, GENE THERAPEUTIC COMPOSITION INCLUDING IT AND USE THEREOF

(71) Applicant: National Cheng Kung University, Tainan (TW)

(72) Inventors: Jeng-Shiung JAN, Tainan (TW); Yu-Fon Chen, Tainan (TW)

(21) Appl. No.: 19/321,456

(22) Filed: Sep. 8, 2025

Related U.S. Application Data
(60) Provisional application No. 63/694,210, filed on Sep. 13, 2024.

(51) Int. Cl. A61K 48/00; A61K 47/64

(52) U.S. Cl. CPC A61K 48/0041; A61K 47/6455

(57) ABSTRACT

Provided in the present invention is a peptide-type nucleic acid carrier, a gene therapeutic composition including it and a use thereof, wherein the peptide-type nucleic acid carrier comprises one or multiple copolypeptide chains comprising: a hydrophilic peptide segment and a hydrophobic peptide segment disposed at C-terminus of the hydrophilic peptide segment. The hydrophilic peptide segment is consisted of a plurality of positively charged amino acids. With the positively charge, the hydrophilic peptide segment attracts nuclear fragments and form a copolypeptide-nucleic acid complex, enabling nuclear fragments' delivery to target cells. On the other hand, the hydrophobic peptide segment protects the nuclear fragments from degradation by circulatory enzymes or intracellular endosome, thereby enhancing transfection efficiency. In light of this, the present invention provides an innovative means of gene therapy.

### CROSS-REFERENCE TO RELATED APPLICATION

This application claims the benefit of U.S. Patent application Ser. No. 63/694,210, filed Sep. 13, 2024. The disclosure of the above application is incorporated herein in its entirety by reference.

### FIELD OF THE INVENTION

The present invention relates to a copolypeptide, particularly relating to a copolypeptide consisting of peptide fragments with different electrical charges, thereby enabling the copolypeptide to carry nucleic acid fragments. The present invention also relates to a nucleic acid fragment delivery system, particularly a system delivering a lipid bilayer membrane relying on charge character of the copolypeptide. The present invention further relates to a method of gene therapy using the copolypeptide to deliver nucleic acid fragments capable of suppressing or knocking out pathogenic genes into a potential transforming cell or a transformed cell.

### BACKGROUND OF THE INVENTION

Conventional cancer treatments, including chemotherapy, surgery, and radiation therapy, are efficacious in eliminating cancer cells but often inflict collateral damage on healthy tissues. Consequently, the development of therapies that precisely target cancer cells is imperative for enhancing patient outcomes and quality of life. Gene therapy, which introduces therapeutic genes into cancer cells or tissues to induce cell death or inhibit tumor growth, holds immense promise for revolutionizing cancer treatment. By directly delivering corrective genetic material to target cells, this approach enables the expression of therapeutic proteins or the upregulation of cancer-combating proteins, ideally achieving a durable therapeutic effect with a single administration. However, efficient delivery of genetic cargo remains a significant challenge.

Naked nucleic acids are susceptible to enzymatic degradation in the bloodstream and are repelled by the cell membrane due to electrostatic repulsion. Therefore, the development of effective gene delivery vehicles is crucial for safeguarding genetic material and facilitating cellular uptake. Viral and non-viral vectors are the two primary gene carrier types. While viral vectors exhibit high transfection efficiency, they pose safety concerns due to potential inflammation and immune responses. Moreover, any gene delivery system intended for human use necessitates rigorous evaluation for pathogenicity and toxicity. Non-viral vectors, characterized by their simplicity, biocompatibility, reduced immunogenicity, ease of synthesis, and adaptability, offer a safer alternative for delivering the therapeutic potential of gene therapy to cancer patients.

Breast cancer represents a significant global health burden, claiming the lives of approximately 685,000 women annually. The pivotal role of the HSP90AB1 gene in breast cancer progression is well-documented, and its inhibition has demonstrated efficacy in suppressing tumor growth and metastasis in other cancer types. However, the therapeutic potential of targeting HSP90AB1 specifically in breast cancer remains largely untapped. RNA interference (RNAi) emerges as a promising strategy to address this unmet clinical need. This powerful technique, mediated by small interfering RNA (siRNA), offers precise and efficient gene silencing by degrading target mRNA transcripts.

The remarkable specificity of RNAi, coupled with its minimal off-target effects, positions it as a highly attractive therapeutic approach for various diseases, including cancer. To sustain gene knockdown, short hairpin RNA (shRNA) can be employed to continuously generate siRNA within cells. By harnessing the potential of RNAi to inhibit HSP90AB1, innovative therapies may be developed to improve outcomes for breast cancer patients. The primary objective was to establish a simple and safe method for delivering gene silencing agents to three-dimensional cancer organoids, ultimately inhibiting tumor cell proliferation. The successful development of such systems is critical for advancing the field of gene therapy for cancer treatment.

### SUMMARY OF THE INVENTION

Peptides have emerged as promising non-viral vectors for DNA delivery, excelling in condensing DNA into nanoparticles and encapsulating specific payloads. Beyond linear polypeptides, branched architectures such as non-linear, star, and dendritic configurations offer innovative avenues to overcome challenges in developing safe and efficient gene delivery systems. Branched copolypeptide featured of highly branched and three-dimensional structures exhibits unique multivalent properties, creating core-shell nanostructures capable of encapsulating substantial drug or vector payloads. The cationic linear poly(L-lysine) (PLL) is a notable peptide-based gene vector, but suffers from low ability to escape from endosomal degradation and low transfection efficiency.

Hereinafter, the present invention focuses on the development of non-viral gene vectors based on copolypeptides, and provides a peptide-type nucleic acid carrier, comprising a copolypeptide chain, wherein the copolypeptide chain comprises: a hydrophilic peptide segment consisting of 5 to 20 substituted or non-substituted amino acids selecting from a group consisting of L-Lysine, L-Arginine, L-Histidine, L-Ornithine and L-Homoarginine; and a hydrophobic peptide segment, disposed at C-terminus or N-terminus of the hydrophilic peptide segment, consisting of 5 to 20 substituted or non-substituted amino acids selecting from a group consisting of L-Alanine, L-Valine, L-Tryptophan, L-Tyrosine, and L-Methionine.

Generally adopted in various embodiments, the peptide-type nucleic acid carrier is a branched copolypeptide comprising at least two the copolypeptide chains, wherein N-terminus of any one of the copolypeptide chains links to a side chain of any one of amino acid residues of the hydrophilic peptide segment or the hydrophobic peptide segment of the other of the copolypeptide chains; or the peptide-type nucleic acid carrier is a star-shaped copolypeptide comprising a core and at least three the copolypeptide chains radially extending from the core, wherein N-terminus or C-terminus of the hydrophilic peptide segment of every one of the copolypeptide chains links to the core.

In preferred embodiments, the hydrophilic peptide segment is consisting of substituted or non-substituted amino acids comprising L-Lysine or L-Homoarginine; the hydrophobic peptide segment is consisting of substituted or non-substituted amino acids comprising L-Alanine or L-Valine.

Provided in another aspect of the present invention is a gene therapeutic composition comprising: the aforementioned peptide-type nucleic acid carrier; and a nucleic acid fragment docking on the peptide-type nucleic acid carrier via electrostatic interaction between a negatively-charged phosphate group and the hydrophilic peptide segment.

Generally adopted in various embodiments, the nucleic acid fragment encodes a CRISPR, a siRNA, a shRNA, a miRNA, a RNAi, an antisense RNA, a nuclease, or a DNase.

Provided in yet one another aspect of the present invention is a method of delivering a nucleic fragment, comprising: combining the aforementioned peptide-type nucleic acid carrier and a nucleic acid fragment to obtain a copolypeptide-nucleic acid complex; and contacting the copolypeptide-nucleic acid complex to a phospholipid bilayer membrane of a subject in need, wherein: as the hydrophobic peptide segment and a hydrophilic surface area of the phospholipid bilayer membrane mutually repulse each other, and the hydrophobic peptide segment creates hydrophobic effect with a hydrophobic area on internal side of the phospholipid bilayer membrane, the copolypeptide-nucleic acid complex enters the phospholipid bilayer membrane; the copolypeptide-nucleic acid complex moves along a concentration gradient across two sides of the phospholipid bilayer membrane, thereby making the copolypeptide-nucleic acid complex penetrate the phospholipid bilayer membrane and enter the subject in need.

In the present invention, ligation of the hydrophobic peptide segment to the hydrophilic peptide segment not only enhances biocompatibility of the peptide-type nucleic acid carrier, but also improves transfection efficiency. In some embodiments exemplified of star-shaped copolypeptide, ligation of poly(L-Alanine) demonstrates excellent biocompatibility when compared to ligation of hydrophobic peptide segment such as poly(L-Phenylalanine) or poly(L-Leucine). As a result of this, the star-shaped copolypeptide shows less hemolytic effects and cytotoxicity, thereby reducing risks of killing healthy cells during circulatory delivery inside the subject in need and avoiding off-target effect.

The peptide-type nucleic acid carrier provided in the present invention also demonstrates superior transfection efficiency on a low N/P ratio condition. As acknowledged so far, transfection efficiency of the branched copolypeptide correlates to the number of side chains in a positive manner. Namely, the larger the number of side chains, the higher the density of functional groups, and, thus, the branched copolypeptide present a more powerful transmembrane delivery function. However, the peptide-type nucleic acid carrier not only achieves at least 70% transfection efficiency in 3-dimensional cancer organoids, but even on a condition of low N/P ratio, each unit of the copolypeptide-nucleic acid complex is capable of delivering a larger amount of nucleic acid fragments. The cancer organoid simulates tissue nature in a 3-dimensional structure and function more precisely, thereby highlighting more authentic therapeutic effects of the copolypeptide-nucleic acid complex. By this approach, development of gene therapy would be accelerated, and a more reliable testing platform is provided for clinic research.

### BRIEF DESCRIPTION OF THE DRAWINGS

FIG. 1A is a schematic illustration of a linear copolypeptide;

FIG. 1B is a schematic illustration of a branched copolypeptide;

FIGS. 1C to 1F are schematic illustrations of star-shaped copolypeptides;

FIGS. 2A to 2B are schematic illustrations of copolypeptide-nucleic acid complexes;

FIG. 3A is a schematic illustration of synthesis of copolypeptides in examples 1 to 6;

FIGS. 3B to 3G are ¹H NMR spectrums to illustrate characterization of chemical structures of copolypeptides in Embodiments 1 to 6 dissolved in THF;

FIG. 4A is a schematic illustration of synthesis of copolypeptides in Embodiments 7 to 9;

FIGS. 4B to 4D are schematic illustrations of chemical structures of copolypeptides with 6, 12 and 24 arms, respectively;

FIGS. 4E to 4M are ¹H NMR spectrums to illustrate characterization of chemical structures of copolypeptides prepared in Embodiments 7 to 9 dissolved in various solvents α-TFA·D₂O or DMSO;

FIGS. 5A to 5C are transmission electronic microscopic images to demonstrate morphologies and sizes of copolypeptide-nucleic acid complexes combining copolypeptides obtained in Embodiments 7 to 9 with pEGFP-C1 plasmids;

FIGS. 6A to 6C are agarose gel electrophoretic images illustrating condensation degrees of plasmid DNA combined with copolypeptides prepared in Embodiments 7 to 9;

FIGS. 7A to 7B illustrate in vitro hemolysis tests in Experimental Example 2;

FIGS. 8A to 8B illustrate organoid cytotoxic test results in Experimental Example 3.1 based on cell lines including NIH/3T3 and BEAS-2B, respectively;

FIGS. 9A to 9C illustrate organoid cytotoxic test results in Experimental Example 3.2 based on cell lines including BEAS-2B, HEK293T and H1299, respectively;

FIGS. 10A to 10D illustrate testing results of transfection efficiency of the copolypeptide-nucleic acid complexes in planar cell cultures in in vitro DNA transfection experiments of Experimental Example 4, where scale bar in the FIG. 10A presents 100 μm;

FIGS. 11A to 11D illustrate testing results of transfection efficiency of the copolypeptide-nucleic acid complexes in organoid cell cultures in in vitro DNA transfection tests of Experimental Example 4, where scale bar in the FIG. 11D presents 200 μm; and

FIGS. 12A to 12B illustrate immunoblotting results of in vitro shRNA plasmid transfection efficiency in Experimental Example 5, including suppression of breast cancer proto-oncogene HSP90 AB1 and activation of apoptotic marker gene PARP, respectively.

### DETAILED DESCRIPTION OF THE INVENTION

The following embodiments are provided to further illustrate the present invention in detail. Upon reviewing the contents of this specification, a person of ordinary skill in the art will readily appreciate the advantages and technical effects of the present invention, and will be able to implement the disclosure or apply it in various alternative embodiments. Accordingly, modifications, substitutions, or alterations to the embodiments described herein may be made without departing from the spirit and scope of the present invention. Moreover, any element or method disclosed in the present invention may be combined with any other element or method described in any of the embodiments without limitation, unless expressly stated otherwise.

As used herein, the singular forms “a,” “an,” and “the” include plural referents unless expressly and unequivocally limited to one referent. The term “or” is used interchangeably with the term “and/or” unless the context clearly indicates otherwise.

The terms “peptide(s),” “polypeptide(s)” and “copolypeptide(s)” are used herein to designate a series of amino acid residues that have multiple amino acid residues, such as 15 to 40 amino acids in length or 15 to 30 amino acids in length, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length. “Copolypeptide(s)” refers to a peptide structure comprising one or multiple amino acid segments. The terms “amino acid segment” and “peptide segment” as used herein, unless defined otherwise, are interchangeable, wherein the peptide segment is consisting of: a repeat of one type of amino acid; or a repeat of amino acids with a single or similar hydrophilic/hydrophobic property.

Peptides, polypeptides, or copolypeptides (hereinafter referred to as peptides) in the present application may include the standard 20 α-amino acids that are used in protein synthesis by cells (i.e. natural amino acids), as well as non-natural amino acids (may be found in nature, but not used in protein synthesis by cells, e.g., orithine, citrulline, and sarcosine, or may be chemically synthesized), amino acid analogs, and peptidomimetics. The amino acids may be D- or L-optical isomers. The peptides may be formed by a condensation or coupling reaction between the α-carbon carboxyl group of one amino acid and the amino group of another amino acid. The terminal amino acid at one end of the chain (amino terminal) therefore has a free amino group, while the terminal amino acid at the other end of the chain (carboxy terminal) has a free carboxyl group. Alternatively, the peptides may be linear or non-linear structure, where linear structure includes branched peptides or cyclic peptides. Moreover, the peptides may optionally be modified or protected with a variety of functional groups or protecting groups, including on the amino and/or carboxy terminus.

The amino acid residues in the peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The copolypeptide in the present application carries hydrophilic area and hydrophobic area, defined by a combination of hydrophilic amino acid residues and hydrophobic amino acid residues. Such configuration of the hydrophilic area and the hydrophobic area renders the copolypeptide capable of carrying a nucleic acid fragment and transporting across a phospholipid bilayer membrane. As such, the copolypeptide may serve as a nucleic acid carrier, or a nucleic acid nanoparticle having a core-shell structure by combining with a nucleic acid.

The term “nucleic acid fragment” used herein, unless defined otherwise, refers to nucleic acid sequence encoding a specific gene, and such nucleic acid fragment may be a DNA fragment or an RNA fragment. The nucleic acid fragment may be chemically modified, including functional group modification such as methylation or deamination.

The term “gene suppression” used herein refers that, compared to a control group without any nucleic acid fragment encoding a nucleic acid sequence targeting a specific gene, a nucleic acid sequence targeting to a specific gene, as encoded by a nucleic acid fragment delivered by using the copolypeptide as a nucleic acid carrier, generates one or multiple siRNA, shRNA, miRNA, or antisense RNA via the intracellular translation system to hybridize with mRNA of the specific gene, resulting in degradation of the mRNA. Alternatively, one or multiple mRNA is generated, based on the nucleic acid fragment, to produce CRISPR, nuclease or DNAase to excise/incise or to degrade mRNA of the specific gene. In some embodiments, the nucleic acid fragment suppresses breast cancer gene expression by producing siRNA, shRNA, miRNA, or antisense RNA, but not limited to this. Gene suppression effects can be evaluated via testing technologies well-acknowledged by a person skilled in the art, as demonstrated in the embodiments of the present application.

The terms “optional” or “optionally” used herein mean that the subsequently described circumstance may or may not occur, and that the description includes embodiments where the circumstance occurs and embodiments where the circumstance does not occur.

The term “about” used herein, when referring a numerical value, is intended to encompass variations of ±20%, ±10%, ±5%, ±1%, ±0.5% or ±0.1% from the stated numerical value. Such variations may result from experimental error, typical measurement error, processing of a compound, composition, concentrate or formulation, variations in the source, manufacture, starting materials or purity of the ingredients used in the present application, or other similar considerations.

The terms “comprise,” “comprising,” “include,” “including,” “have,” “having,” “contain,” “containing,” and any other variations thereof used herein are intended to denote a non-exclusive inclusion. For example, when an object is described as “comprising” a limitation, unless otherwise expressly specified, it may additionally include other ingredients, elements, components, structures, regions, parts, devices, systems, steps, or connections, and should not be construed as excluding the presence of other limitations.

The gene therapeutic composition provided in the present invention may be administered intravenously, intra-arterially, intraperitoneally, intramuscularly, intradermally, intratumorally, orally, epidermally, intranasally, on the cheeks, intrarectally, enterally, intravaginally, via inhalation, or topically.

The gene therapeutic composition provided in the present invention may be administered to a subject including an animal; the terms “subject”, “object”, or “patient” can be interchangeable. The stated animal may be a fish, a bird or a mammal, but not limited to this. The stated animal may be exemplified by a cat, a canis, a bovine, a horse, a swine, or a human, but not limited to this.

The term “effective amount” used herein is intended to describe an administered amount of any nucleic acid fragment, and, with the stated administered amount, no matter the nucleic acid fragment is administered individually, or in combination with other types of drug or therapeutic agent, the nucleic acid fragment achieves to protect the subject by slowing down disease progression or removing the cause of the disease, and to minimize severity of the disease symptoms, to maximize frequency or duration of an asymptomatic period, or to avoid damage or disorder caused by the disease. The ability to slow down disease progression may be evaluated by technologies well-acknowledged in the art, such as predicting therapeutic effects in a human body via cell models, animal models or clinical trials.

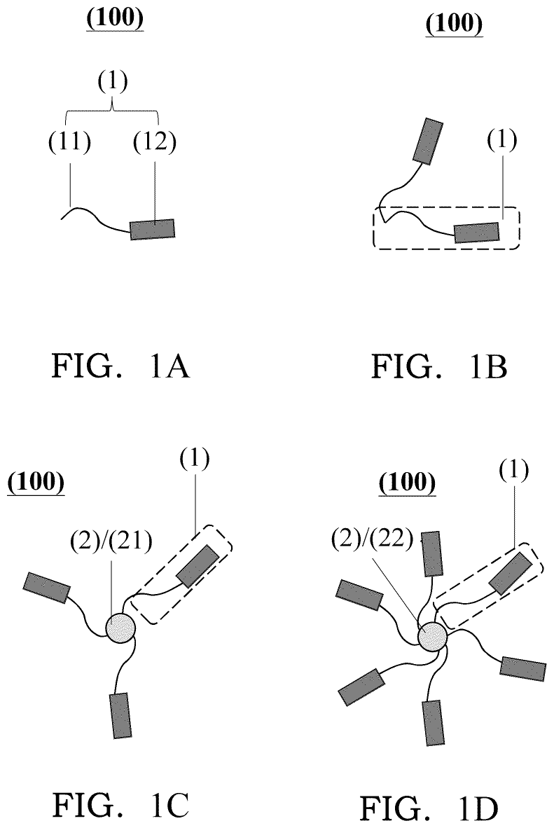

Provided in the first aspect of the present invention is a peptide-type nucleic acid carrier (100), as shown in FIG. 1A, comprising a copolypeptide chain (1), wherein the copolypeptide chain (1) comprises:
a hydrophilic peptide segment (11) consisting of 5 to 20 substituted or non-substituted amino acids selecting from a group consisting of L-Lysine, L-Arginine, L-Histidine, L-Ornithine and L-Homoarginine; and
a hydrophobic peptide segment (12), disposed at C-terminus or N-terminus of the hydrophilic peptide segment, consisting of 5 to 20 substituted or non-substituted amino acids selecting from a group consisting of L-Alanine, L-Valine, L-Tryptophan, L-Tyrosine, and L-Methionine. Preferably, the C-terminus of the hydrophobic peptide segment (12) links to the N-terminus of the hydrophilic peptide segment (11).

Generally adopted in various embodiments, a ratio of a length of the hydrophilic peptide segment (11) to a length of the hydrophobic peptide (12) segment is (2 to 4): 1, preferably 2:1, 3:1 or 4:1, and more preferably 2:1.

In some embodiments, the hydrophilic peptide segment (11) is consisting of substituted or non-substituted amino acids comprising L-Lysine or L-Homoarginine. Preferably, the hydrophilic peptide segment (11) may be selected from a group consisting of L-lysines, L-lysine₆, L-lysine₇, L-lysines, L-lysine₉, L-lysine₁₀, L-lysine₁₂, L-lysine₁₅, L-lysine₂₀, L-homoarginine₅, L-homoarginine₆, L homoarginine₇, L-homoarginine₈, L-homoarginine₉, L-homoarginine₁₀, L-homoarginine₁₂, L-homoarginine₁₅, and L-homoarginine₂₀. More preferably, the hydrophilic peptide segment (11) is selected from L-lysine₁₀ and L-lysine₂₀.

In some embodiments, the hydrophobic peptide segment (12) is consisting of substituted or non-substituted amino acids comprising L-Alanine or L-Valine. Preferably, the hydrophobic peptide segment (12) is selected from a group consisting of L-alanine₅, L-alanine₆, L-alanine₇, L-alanine₈, L-alanine₉, L-alanine₁₀, L-alanine₁₂, L-alanine₁₅, L-alanine₂₀, L-valines, L-valine₆, L-valine₇, L-valine₈, L-valine₉, L-valine₁₀, L-valine₁₂, L-valine₁₅ and L-valine₂₀. More preferably, the hydrophobic peptide segment (12) is selected from L-alanine₅ and L-alanine₁₀.

Compared to linear copolypeptide, branched copolypeptide is able to increase density of functional groups on the side chains, thereby promoting nucleic acid condensation and improving transfection efficiency. To this end, in some embodiments, as shown in FIG. 1B, the peptide-type nucleic acid carrier (100) may be a branched copolypeptide comprising at least two the copolypeptide chains (1), wherein N-terminus of any one of the copolypeptide chains (1) links to a side chain of any one of amino acid residues of the hydrophilic peptide segment (11) or the hydrophobic peptide segment (12) of the other of the copolypeptide chains (1), but one amino acid residue at C- or N-terminus of the hydrophilic peptide segment (11) is excluded.

In some other embodiments, as shown in FIGS. 1C to 1F, to further increase density of functional groups on the side chains, the peptide-type nucleic acid carrier (100) may be a star-shaped copolypeptide comprising a core (2) and at least three the copolypeptide chains (1) radially extending from the core, wherein N-terminus or C-terminus of the hydrophilic peptide segment (11) of every one of the copolypeptide chain (1) links to the core (2). Preferably, C-terminus of the hydrophilic peptide segment (11) of every one of the copolypeptide chain (1) links to the core (2). In the spatial structure of the star-shaped copolypeptide, the hydrophobic peptide segments (12) surround the hydrophilic peptide segments (11) so that, when a nucleic acid fragment (200) binds to one or multiple of the hydrophilic peptide segments (11), the negative charge of the nucleic acid fragment (200) is shielded by the hydrophobic peptide segments (12). In this way, mutual repulsion between the negative charges of the nucleic acid fragment (200) and the negatively-charged phosphate groups on the outer side of the phospholipid bilayer membrane can be minimized during passing through the phospholipid bilayer membrane, thereby enhancing transfection efficiency.

In preferred embodiments, the star-shaped copolypeptide comprises at least 3 to 24 of the copolypeptide chains (1), such as 3, 6, 12, or 24 the copolypeptide chains (1), or 4, 8, 16 of the copolypeptide chains (1). Concretely, the number of the copolypeptide chains (1) is determined by a number of coordination legs on the core (2). More preferably, the star-shaped copolypeptide comprises 6 of the copolypeptide chains (1).

In the preferred embodiments, the core (2) may be a 3-leg core (21), a 6-leg core (22), a 12-leg core (23) or a 24-leg core (24). Exemplarily, the 3-leg core (21) may be 1,1,1-Tris(hydroxymethyl) propane or tris(2-aminoethyl)amine; the 6-leg core (22) may be dipentaerythritol, G1 bis-MPA-OH or G1 polyglycerol dendrimer; the 12-leg core (23) may be G2 bis-MPA-OH or G2 polyglycerol dendrimer; the 24-leg core (24) may be G3 bis-MPA-OH or G3 polyglycerol dendrimer, but not limited to this. Preferably, the core (2) is the 6-leg core (22) comprising G1 polyglycerol dendrimer.

In some preferred embodiments, the core (2) may be other types of dendrimers such as PAMAM dendrimers, polyglutamine dendrimers, polypropyleneimine dendrimers, or polyester dendrimers, and the core (2) may be a 4-leg core, a 8-leg core or a 16-leg core, but not limited to this.

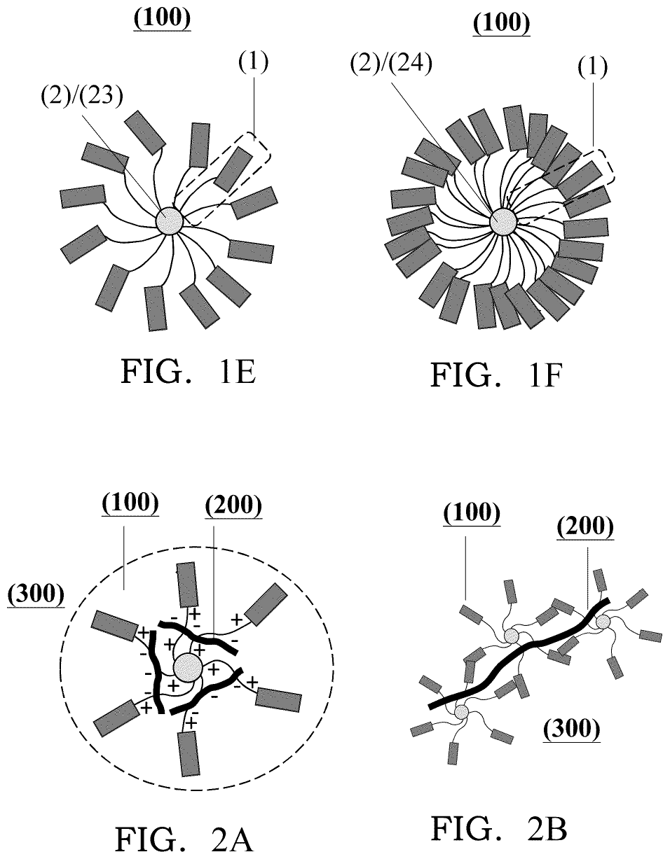

Provided in the second aspect in the present invention is a gene therapeutic composition comprising: the peptide-type nucleic acid carrier (100) in the first aspect in the present invention; and a nucleic acid fragment (200), wherein the nucleic acid fragment (200) docks on the peptide-type nucleic acid carrier (100) via electrostatic interaction between a negatively-charged phosphate group and the hydrophilic peptide segment (11). Taking a 6-leg core (22) as the core (2) for example, as shown in FIGS. 2A to 2B, as being negatively charged, the nucleic acid fragments (200) cluster on the hydrophilic peptide segment(s) (11) with dense positive charges. Based on electrostatic attraction effects, the nucleic acid fragments (200) may further drag the copolypeptide chains (1) in the neighborhood, or may attract other molecules of peptide-type nucleic acid carrier (100) in the neighborhood, and result in clustering effects. Accordingly, the nucleic acid fragments (200) are encapsulated by multiple copolypeptide chains (1) or multiple molecules of the peptide-type nucleic acid fragments (100) to form a nano-scale copolypeptide-nucleic acid complex (300). By this, the hydrophobic peptide segments (12) exposed on the outer surface of the copolypeptide-nucleic acid complex (300) prevents the nucleic acid fragments (200) from enzymatic degradation, and enhances hydrophobic effects during transportation across the phospholipid bilayer membrane, thereby improving transfection efficiency.

Generally adopted in various embodiments, a N/P ratio of the peptide-type nucleic acid carrier (100) to the nucleic acid fragment (200) is 2.3 to 3.3, and preferably 2.5 to 3.1.

Generally adopted in various embodiments, the nucleic acid fragment (200) encodes a CRISPR, a siRNA, a shRNA, a miRNA, a RNAi, an antisense RNA, a nuclease, or a DNase. Preferably, the nucleic acid fragment (200) is a plasmid DNA carrying a proto-oncogene targeting suppressor gene to, wherein the proto-oncogene targeting suppressor gene encodes a siRNA or a shRNA. In one example, the proto-oncogene is a HSP90 AB1 gene, but not limited to this.

Provided in the third aspect in the present invention is a use of the peptide-type nucleic acid carrier (100) for making a gene therapeutic composition for treating breast cancer, wherein the gene therapeutic composition comprises the peptide-type nucleic acid carrier (100) and a nucleic acid fragment (200), and the nucleic acid fragment (200) docks on the peptide-type nucleic acid carrier (100) via electrostatic interaction between a negatively-charged phosphate group and the hydrophilic peptide segment (11). The use further comprises: administering the gene therapeutic composition to a subject in need so that the nucleic acid fragment (200) arrives at an effective amount, and the nucleic acid fragment (200) is incorporated to a nucleic acid translation system of the subject in need to produce sufficient amount of breast cancer gene targeting nucleic fragment.

In some embodiments, the use is to administer the gene therapeutic composition to a breast cancer cell tissue or tumor of the subject in need, wherein the effective amount is at least 1.0 to 5.0 mmol, and preferably 2.0 to 3.0 mmol.

In the preferred embodiment, the breast cancer gene is a HSP90 AB1 gene.

Provided in the fourth aspect of the present invention is a method of delivering a nucleic fragment, comprising: combining the peptide-type nucleic acid carrier (100) in the first aspect of the present invention and a nucleic acid fragment (200) to obtain a copolypeptide-nucleic acid complex (300); and contacting the copolypeptide-nucleic acid complex (300) to a phospholipid bilayer membrane of a subject in need, wherein: as the hydrophobic peptide segment (12) and a hydrophilic surface area of the phospholipid bilayer membrane mutually repulse each other, and the hydrophobic peptide segment (12) creates hydrophobic effect with a hydrophobic area on internal side of the phospholipid bilayer, the copolypeptide-nucleic acid complex (300) enters the phospholipid bilayer membrane; the copolypeptide-nucleic acid complex (300) moves along a concentration gradient across two sides of the phospholipid bilayer membrane, thereby making the copolypeptide-nucleic acid complex (300) penetrate the phospholipid bilayer membrane and enter the subject in need.

Exemplary embodiments of the present invention are further described in the following examples, which should not be construed to limit the scope of the present invention.

### #Preparation of N-Carboxy Anhydride Monomer of Amino Acids

N-carboxy anhydride monomers of Z-L-Lysine, L-Alanine, L-Leucine and L-phenylalanine were prepared by triphosgene reacting with Z-L-Lysine, L-Alanine, L-Leucine and L-phenylalanine in THF. The prepared NCA monomers are designated to be ZLL-NCA, Ala-NCA, Leu-NCA and Phe-NCA.

### Embodiment 1 (Em1)

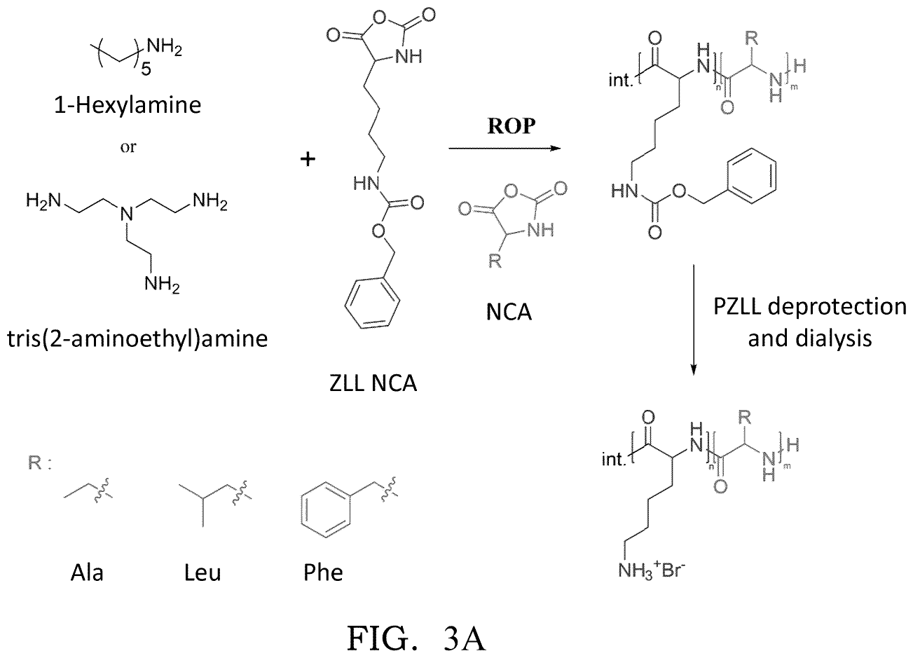

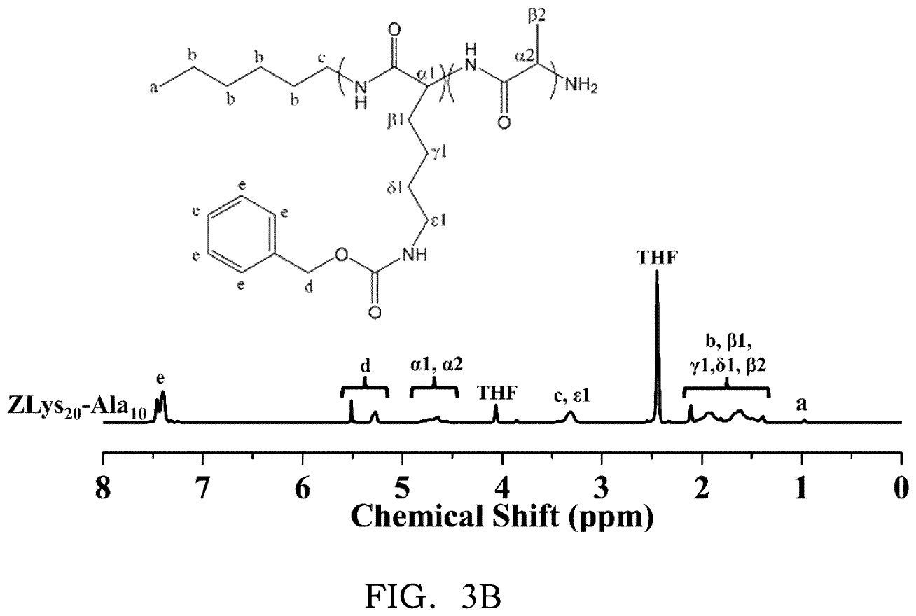

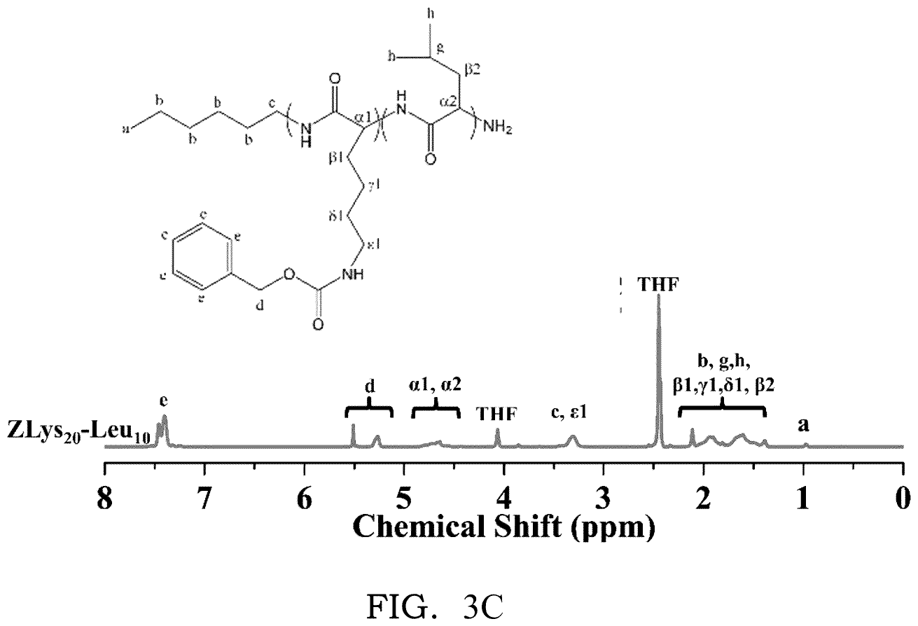

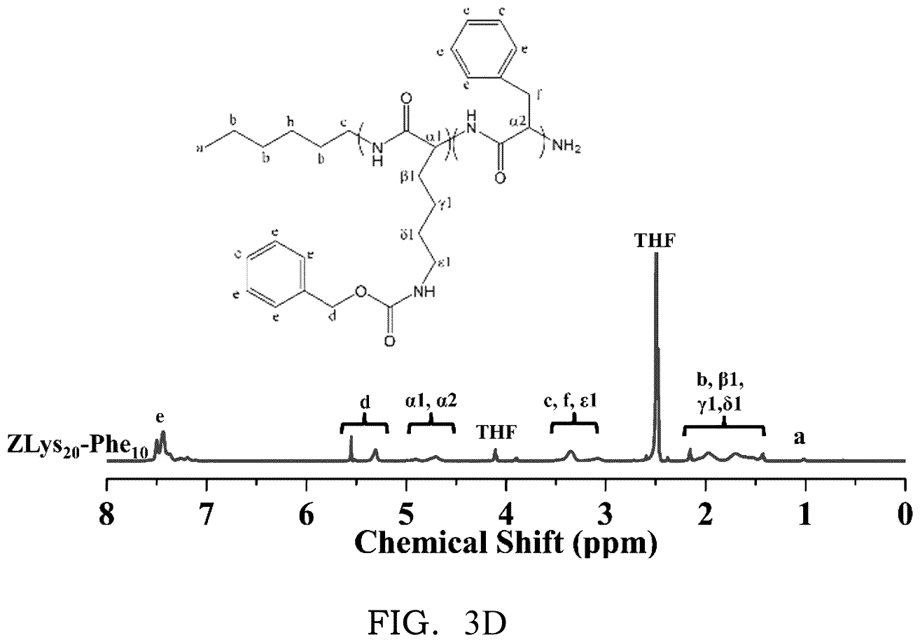

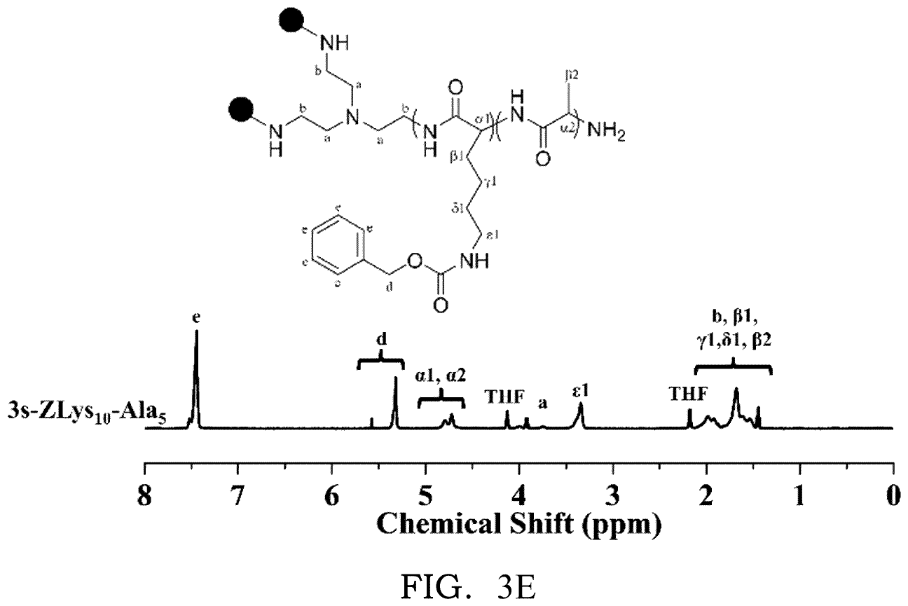

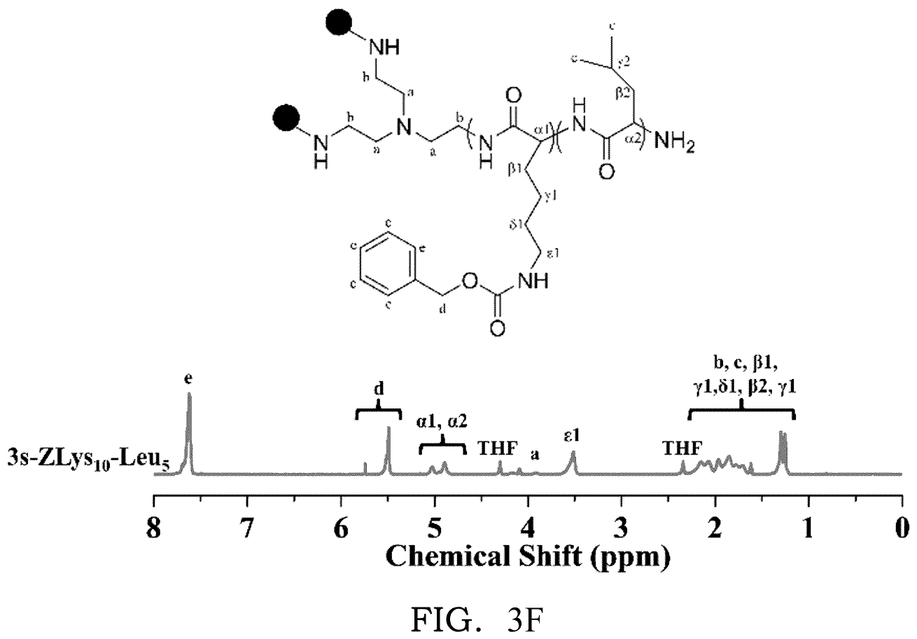

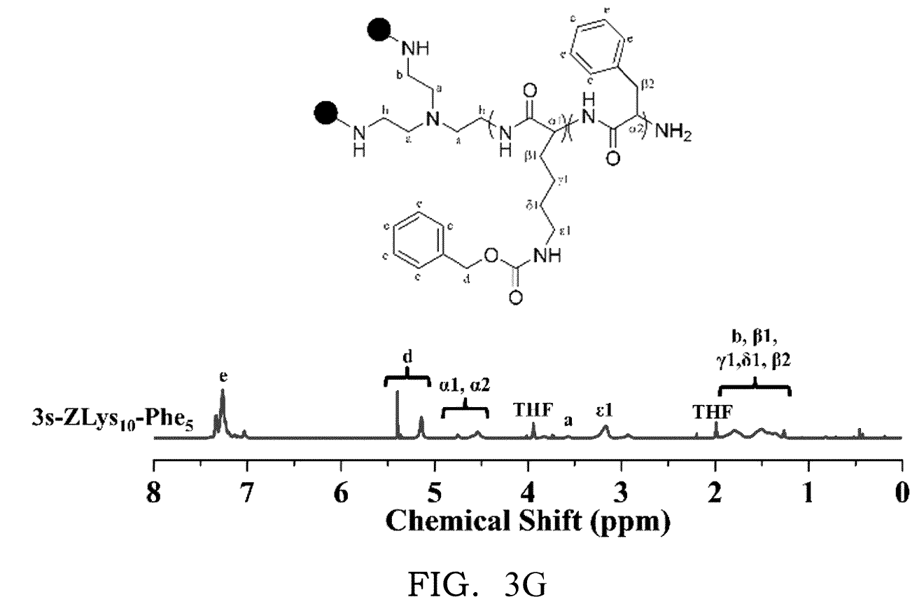

In the Em1, with reference to FIG. 3A, 1-hexylamine was used as the initiator and the molar ratio of the initiator, ZLL-NCAs, Ala-NCAs was set to be 1:20:10. Firstly, 1-hexylamine solution, ZLL-NCA solution, Ala-NCA solution were prepared in THF. After adding a designated amount of the 1-hexylamine solution to the ZLL-NCA solution to prepare a reaction mixture, reaction mixture was stirred at room temperature under argon atmosphere to initiate the ROP reactions for 72 hrs so as to obtain a first reaction solution. After 72 hrs, the designated amount of Ala-NCA solution was added to the first reaction solution and stirred at room temperature to continue ROP reactions for additional 72 hrs, a second reaction solution was obtained containing completely synthesized PZLL-P(Ala) copolypeptide. As shown in FIG. 3B, the structure of PZLL-P(Ala) copolypeptide was identified by ¹H NMR. Finally, the second reaction solution was then added with anhydrous diethyl ether so that the as-synthesized PZLL-P(Ala) was purified by precipitation. The precipitates were collected and the protecting Z group on PZLL segment was removed using hydrogen bromide (HBr, 33 wt % in acetic acid) in trifluoroacetic acid (TFA). The final product linear copolypeptide Lys₂₀-Ala₁₀ was then obtained, followed by precipitation using ethyl ether (>99%, ECHO) and lyophilization after dialysis against DI water.

### Embodiment 2 (Em2)

Processing steps of Em2 are overall the same as those in the Em 1, but the difference lies in using Leu-NCA monomers, and the second reaction solution contained PZLL-P(Leu) copolypeptide. As shown in FIG. 3C, the structure of PZLL-P(Leu) copolypeptide was identified by ¹H NMR, and the final product was linear copolypeptide Lys₂₀-Leu₁₀.

### Embodiment 3 (Em3)

Processing steps of Em3 are overall the same as those in the Em1, but the difference lies in using Phe-NCA monomers, and the second reaction solution contained PZLL-P(Phe) copolypeptide. As shown in FIG. 3D, the structure of PZLL-P(Phe) copolypeptide was identified by ¹H NMR, and the final product was linear copolypeptide Lys₂₀-Phe₁₀.

### Embodiment 4 (Em4)

As depicted in FIG. 3A, processing steps of Em4 are overall the same as those in the Em1, but the difference lies in using tris(2-aminoethyl)amine (Tren) as the initiator, and the molar ratio of Tren, ZLL-NCA, Ala-NCA was set to be 1:30:15. The second reaction solution contained 3s-PZLL-P(Ala) copolypeptide, and the structure of PZLL-P(Phe) copolypeptide was identified by ¹H NMR as shown in FIG. 3E. The final product was star-shaped copolypeptide 3s-Lys₁₀-Ala₅.

### Embodiment 5 (Em5)

Processing steps of Em5 are overall the same as those in the Em4, but the difference lies in using Leu-NCA monomers, and the second reaction solution contained 3s-PZLL-P(Leu) copolypeptide. As shown in FIG. 3F, the structure of PZLL-P(Leu) copolypeptide was identified by ¹H NMR, and the final product was star-shaped copolypeptide 3s-Lys₁₀-Leu₅.

### Embodiment 6 (Em6)

Processing steps of Em6 are overall the same as those in the Em4, but the difference lies in using Phe-NCA monomers, and the second reaction solution contained 3s-PZLL-P(Phe) copolypeptide. As shown in FIG. 3G, the structure of PZLL-P(Phe) copolypeptide was identified by ¹H NMR, and the final product was star-shaped copolypeptide 3s-Lys₁₀-Phe₅.

### Embodiment 7 (Em7)

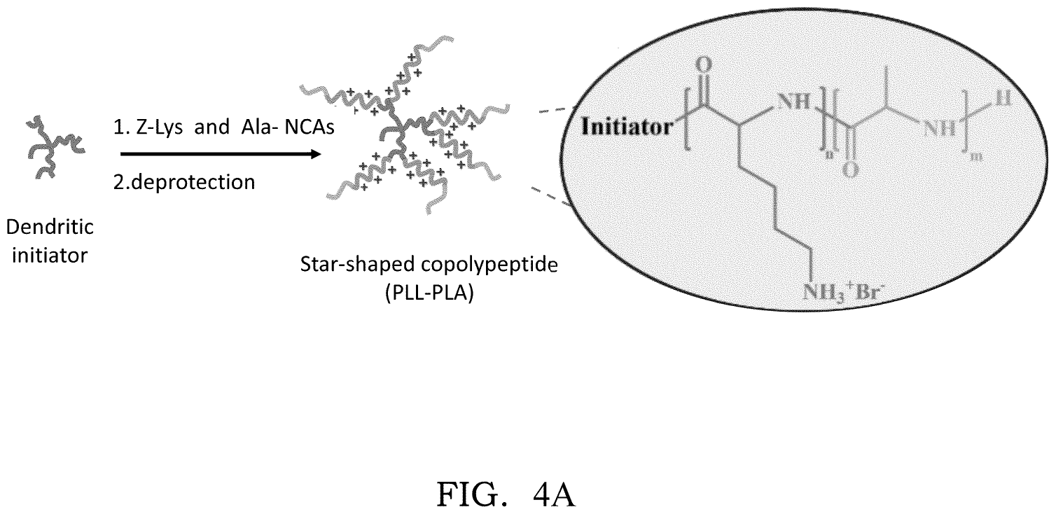

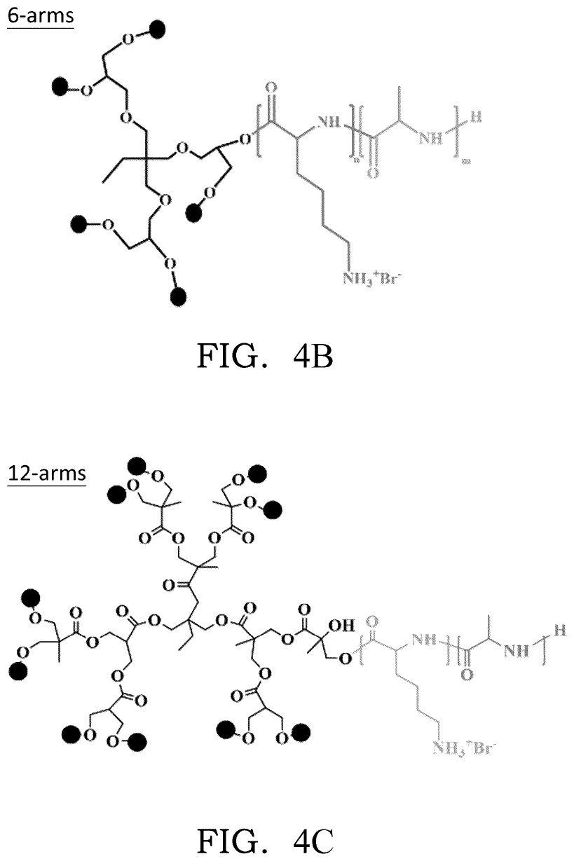

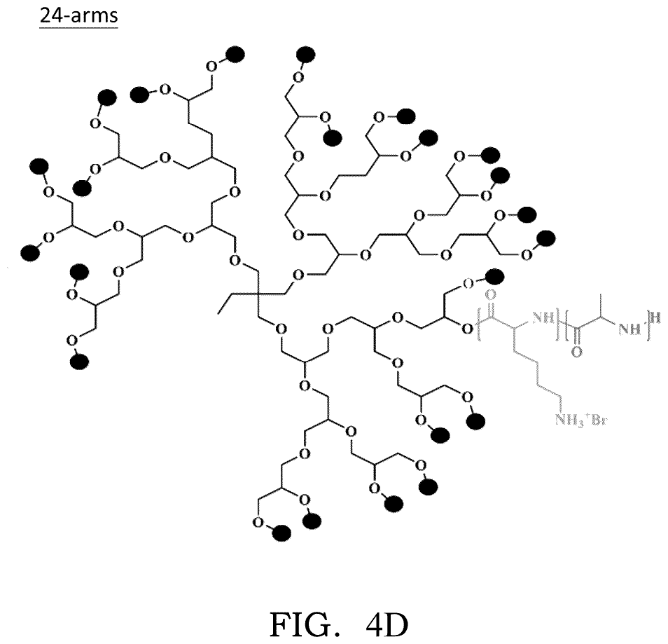

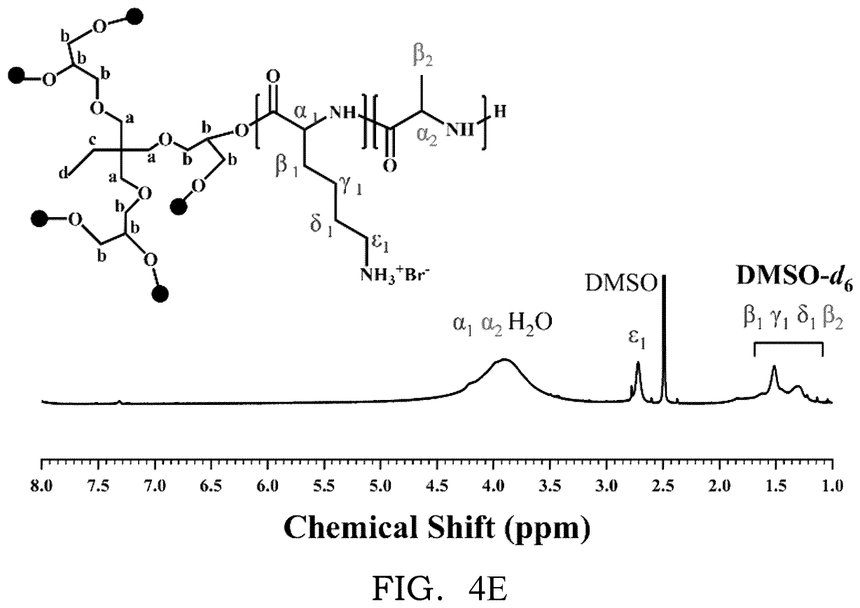

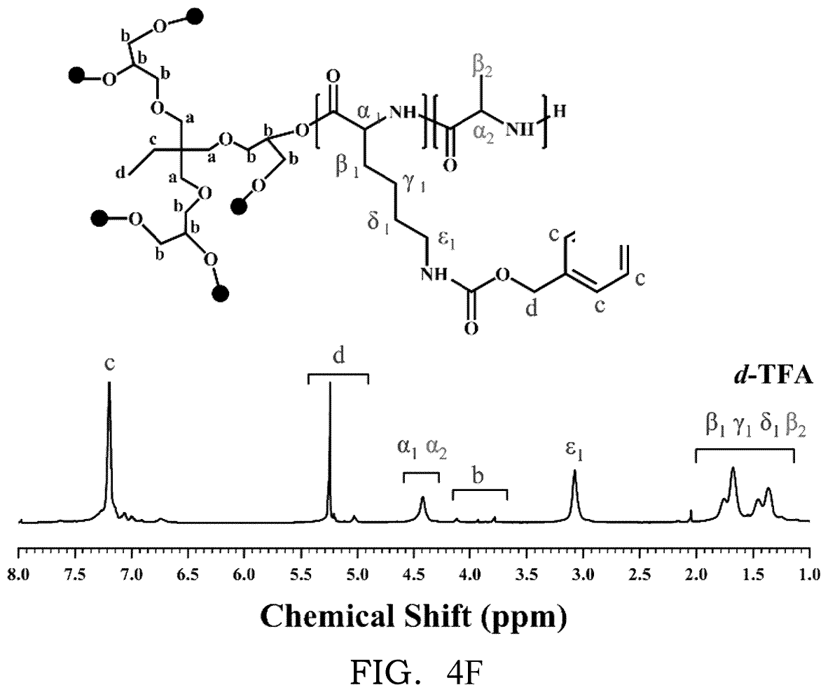

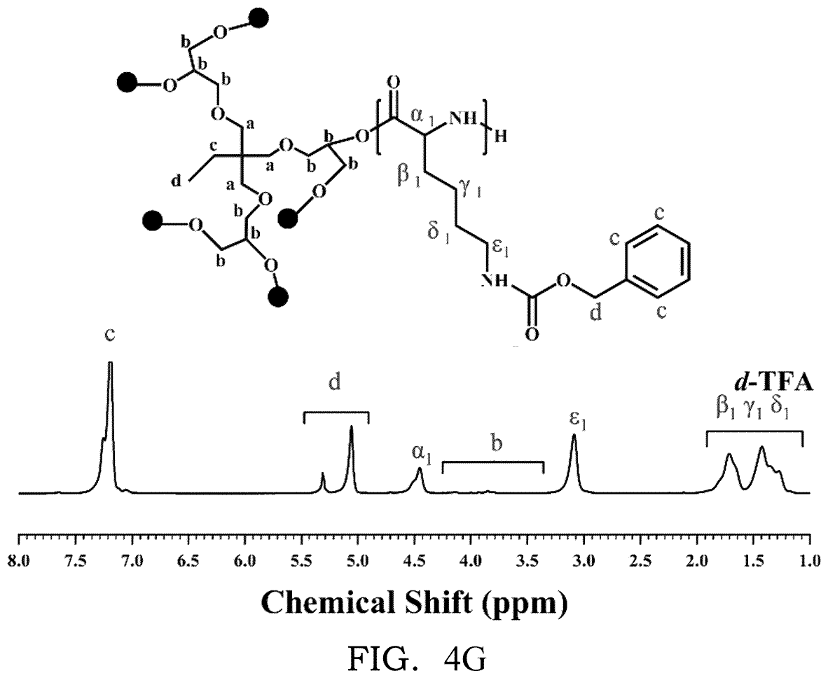

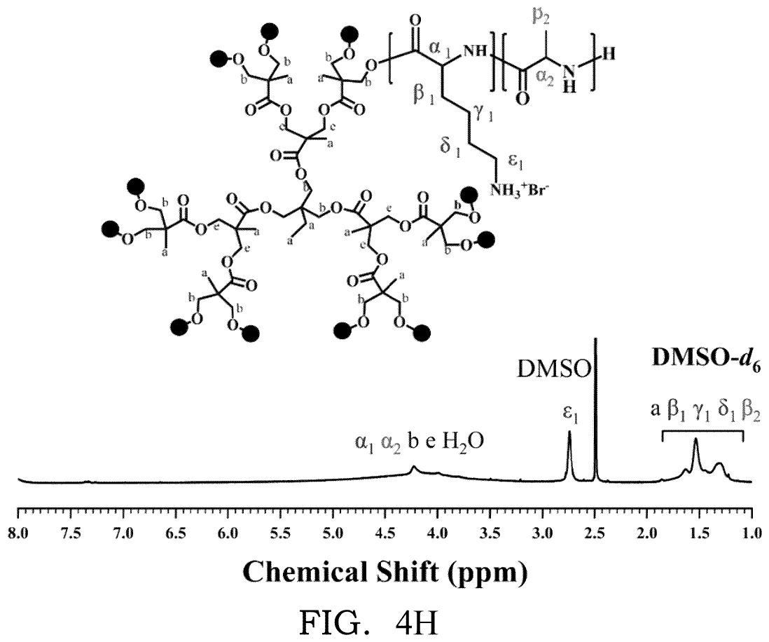

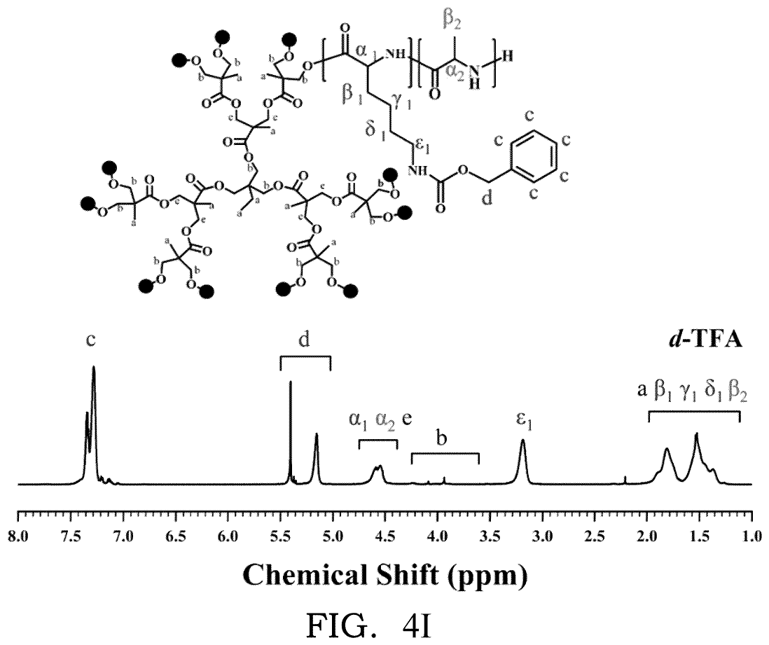

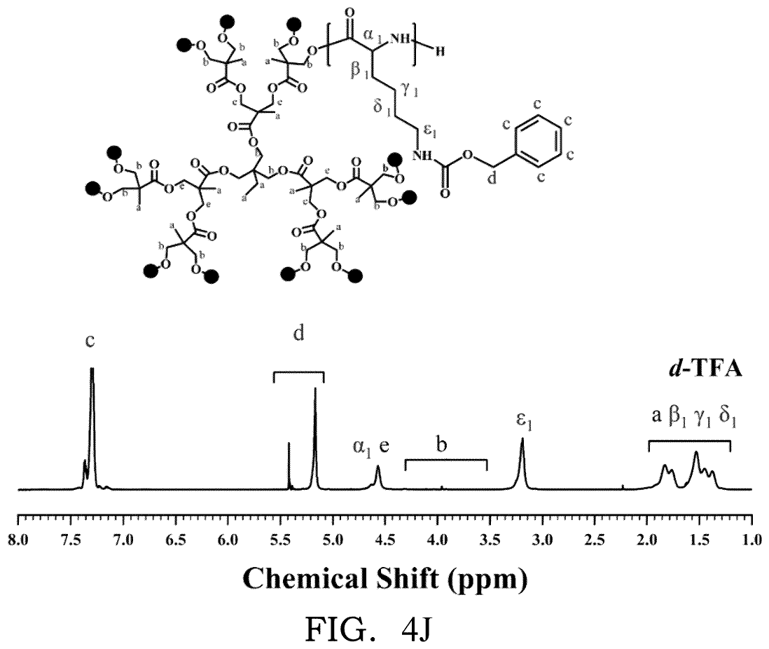

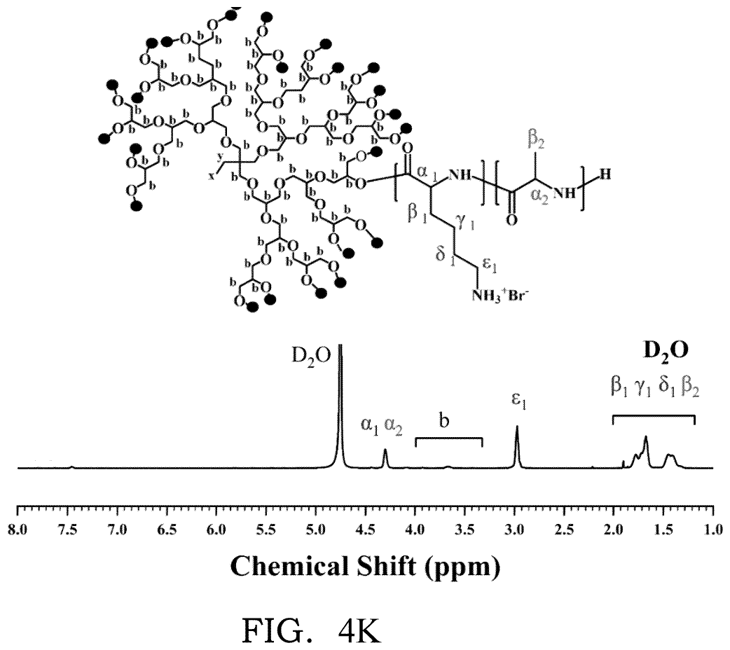

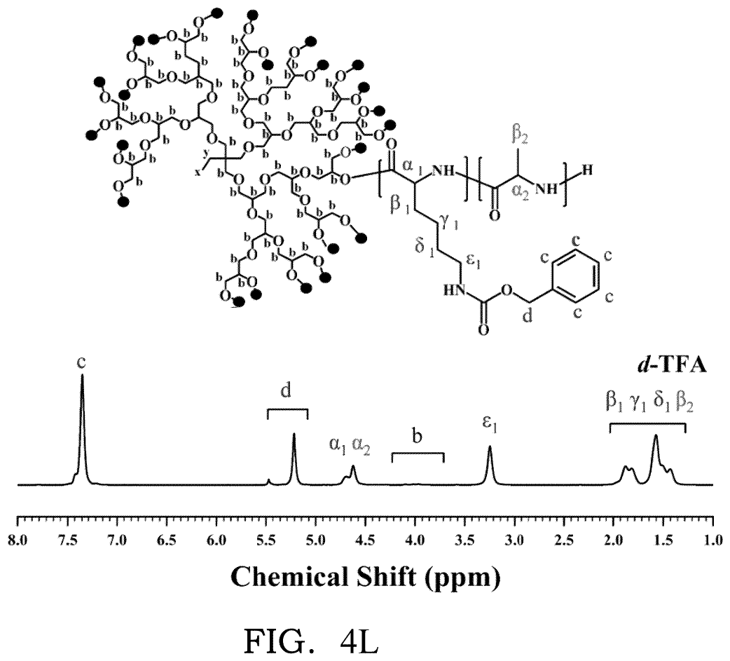

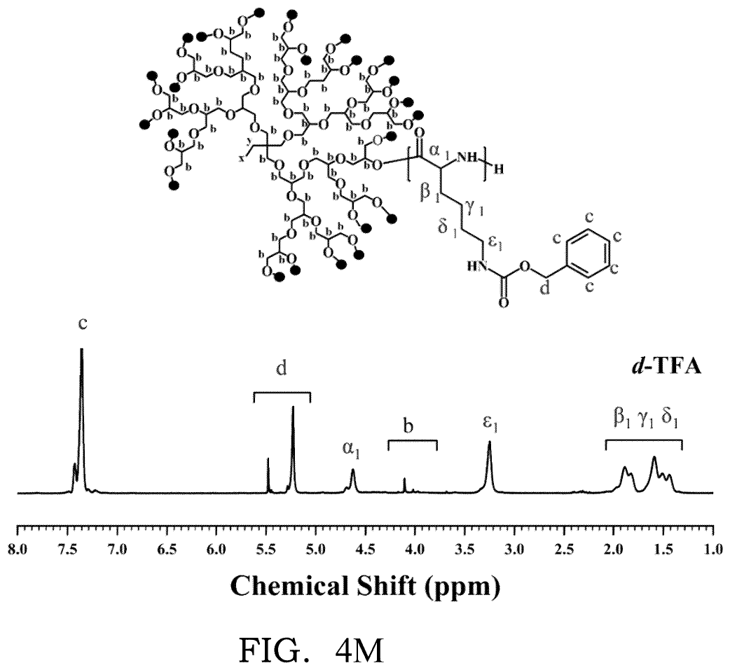

In Em7, the molar ratio of the initiator PGD1, ZLL-NCA, Ala-NCA was adjusted to be 1:60:30. The processing steps were illustrated in FIG. 4A, and chemical structures of star-shaped copolypeptides with 6, 12 and 24 arms were depicted in FIGS. 4B to 4D, respectively.

Firstly, PGD1 solution, ZLL-NCA solution, Ala-NCA solution were prepared in dimethylformamide (DMF), and final concentrations were 0.02 mM, 1.0M and 1.0M, respectively. The PGD1 solution was then treated with a 1,1,3,3-Tetramethylguanidine (TMG) stock solution (0.33 μmol, 54.5 mM in DMF) to achieve a final TMG concentration of 6.0 mM. A first activation solution was obtained after 30 minutes of alcohol group activation. The first activation solution was added to a freshly prepared ZLL-NCA solution (1.0 M in DMF), and stirred at 25° C. under argon atmosphere for ROP reactions for 72 hours so that a first reaction solution was obtained. Subsequently, Ala-NCA solution was introduced to the first reaction solution and stirred for an additional 48 hours under nitrogen to continue ROP reactions. A second reaction solution containing star-shpaed copolypeptide 6s-PZLL₁₀-b-PLA₅ was then obtained. The second reaction solution was dialyzed sequentially against DMF for 1 hour, methanol for 4 hours, and deionized water for 48 hours. Finally, the purified 6s-PZLL₁₀-b-PLA₅ solution was obtained and lyophilized to gain a white solid product.

As the functional groups of NCA monomers can interfere with polypeptide synthesis or NCA stability, Z groups were employed to protect these functional groups. Following polymerization, these protective groups were removed to expose the functional groups. To achieve this, HBr (fivefold molar excess relative to Z groups) was slowly added to a solution prepared with 0.8 g white solid in 40-mL TFA. After stirring for 1 hour at room temperature, the Z groups of 6s-PZLL₁₀-b-PLA₅ were removed and a 6s-PZLL₁₀-b-PLA₅ solution was obtained. The 6s-PZLL₁₀-b-PLA₅ was precipitated by pouring the 6s-PZLL₁₀-b-PLA₅ solution into excess ether (300 mL). The precipitated 6s-PZLL₁₀-b-PLA₅ was collected by centrifugation and dried. The dried solid was dissolved in deionized water and dialyzed against deionized water for 3 days using a cellulose membrane with a molecular weight cutoff (MWCO) of 6-8 kDa or 12-14 kDa. Lyophilization of the dialyzed solution yielded cotton-like products identified as 6s-PLL₁₀-b-PLA₅ via ¹H NMR as shown in FIGS. 4E to 4F.

### Embodiment 8 (Em8)

Processing steps of Em7 are overall the same as those in the Em7, but the difference lies in using bis-MPA-OH dendrimer as the initiator, and the molar ratio of the bis-MPA-OH dendrimer, ZLL-NCA and Ala-NCA was set to be 1:120:60. The second reaction solution contained star-shaped copolypeptide 12s-PZLL₁₀-b-PLA₅. As shown in FIGS. 4H to 4J, the cotton-like product was identified as 12s-PZLL₁₀-b-PLA₅ by ¹H NMR.

### Embodiment 9 (Em9)

Processing steps of Em9 are overall the same as those in the Em7, but the difference lies in using PGD3 as the initiator, and the molar ratio of the PGD3, ZLL-NCA and Ala-NCA was set to be 1:240:120. The second reaction solution contained star-shaped copolypeptide 24s-PZLL₁₀-b-PLA₅. As shown in FIGS. 4K to 4M, the cotton-like product was identified as 24s-PZLL₁₀-b-PLA₅ by ¹H NMR.

### Experimental Example 1: Preparation and Characterization of Copolypeptide-DNA Complex

Copolypeptide solutions (400 μM) were prepared by mixing star-shaped copolypeptides obtained in Em7 to Em9 with predetermined volume of pEGFP-C1 solution (1 μg μL⁻¹) at varying N/P molar ratios to obtain copolypeptide/DNA mixtures. The mixtures were vortexed for 30 seconds and allowed to stand for 30 minutes at room temperature so that copolypeptide-DNA complexes were retrieved. The hydrodynamic diameter (particle size), polydispersity index (PDI), and zeta potential of the copolypeptide-DNA complexes were determined in triplicate (n=3) using an Otsuka ELSZ-1000 dynamic light scattering (DLS) system. Each sample volume was 1.5 mL in purified water, containing a DNA concentration of 5.76 μg mL⁻¹. The morphology of the copolypeptide-DNA complexes was visualized using a Hitachi H-7500 transmission electron microscope (TEM), and a portion of each sample was resolved in agarose gel electrophoresis.

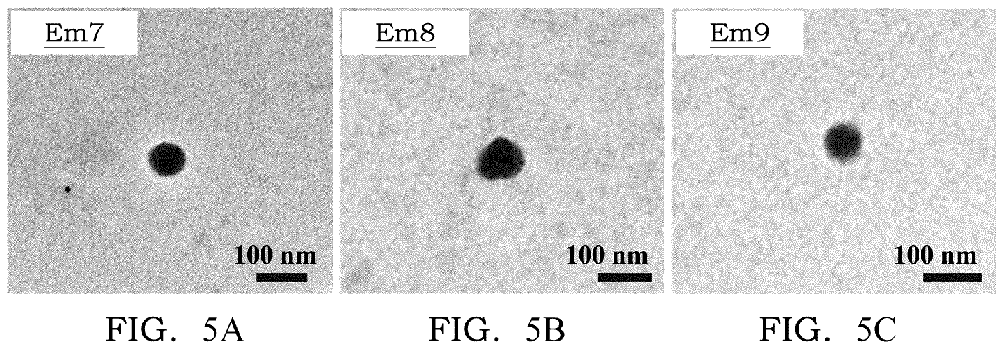

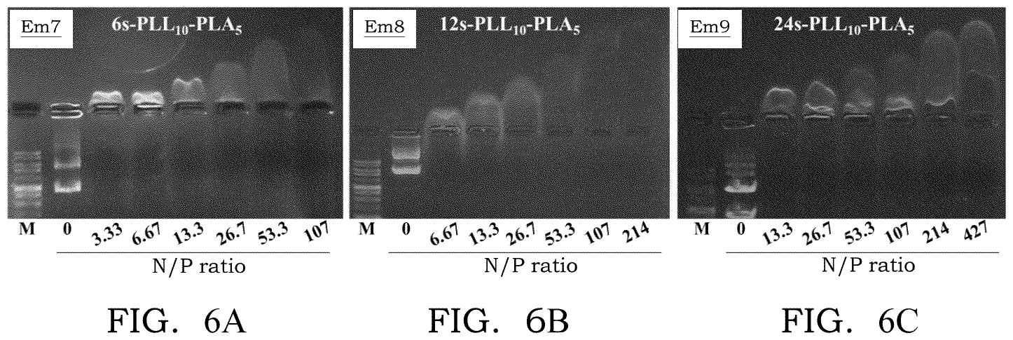

Star-shaped copolypeptide encapsulates the plasmid DNA may promote condensation of plasmid DNA into nano-scale copolypeptide-DNA complexes providing protective effects on the plasmid DNA, and such encapsulation ensures optimal therapeutic efficacy as plasmid DNA can evade degradation by host enzymes in blood circulation, which is crucial for timely DNA release. As shown in Table 1, Zeta-potential measurements confirmed the positive charge of the copolypeptide-DNA complexes at an N/P ratio of 1.34. As indicated in Table 2, Dynamic light scattering (DLS) analysis indicated that the resulting copolypeptide-DNA complexes exhibited unimodal size distributions ranging from 50 to 100 nm. As exhibited in FIGS. 5A to 5C, transmission electron microscopy (TEM) revealed that sizes of copolypeptide-DNA complexes formation at N/P ratios 0.37 (Em7), 1.34 (Em8), and 1.34 (Em9) ranged from 30 to 70 nm. Moreover, agarose gel electrophoresis demonstrated that complete plasmid DNA condensation was achieved at specific N/P ratios for each star-shaped copolypeptide: 3.33 (Em7), 6.67 (Em8), and 13.3 (Em9) as depicted in FIGS. 6A to 6C.

TABLE 1

| N/P ratio | Em7 | Em8 | Em9 |
|---|---|---|---|
| 0.67 | −53.6 ± 11.4 | −0.41 ± 0.1 | −18.2 ± 3.3 |
| 1.34 | 26.47 ± 1.0 | 23.5 ± 2.9 | 7.8 ± 6.218 |
| 2.01 | 37.27 ± 2.9 | 35.5 ± 3.3 | 24.4 ± 7.6 |
| 4.01 | 33.33 ± 1.8 | 51.2 ± 2.7 | 35.0 ± 2.3 |
| 5.35 | 43.13 ± 0.8 | 63.1 ± 5.7 | 38.8 ± 0.7 |

TABLE 2

| | Em7 | | Em8 | | Em9 | |
|---|---|---|---|---|---|---|
| N/P ratio | size (nm) | PDI | size (nm) | PDI | size (nm) | PDI |
| 0.67 | 69.0 ± 2.9 | 0.47 ± 0.10 | 73.7 ± 9.2 | 0.31 ± 0.07 | 70.6 ± 21.5 | 0.31 ± 0.07 |
| 1.34 | 105.2 ± 5.1 | 0.25 ± 0.02 | 77.1 ± 5.5 | 0.30 ± 0.04 | 27.4 ± 7.65 | 0.30 ± 0.04 |
| 2.01 | 90.6 ± 2.9 | 0.24 ± 0.02 | 73.0 ± 7.6 | 0.32 ± 0.03 | 77.1 ± 17.0 | 0.32 ± 0.03 |
| 4.01 | 68.5 ± 1.3 | 0.26 ± 0.02 | 76.8 ± 3.6 | 0.41 ± 0.15 | 88.2 ± 24.2 | 0.41 ± 0.15 |
| 5.35 | 85.6 ± 15.8 | 0.66 ± 0.09 | 73.8 ± 4.0 | 0.31 ± 0.00 | 75.7 ± 5.9 | 0.31 ± 0.00 |

### Experimental Example 2: In Vitro Hemolysis

A 100 μL aliquot of copolypeptide solution was mixed with 100 μL of the hRBC suspension (10 v/v %) and incubated at 37° C. for 1 hour, where the copolypeptide was prepared from Embodiments 1 to 9. Following centrifugation at 1000 rpm for 5 minutes, the supernatant was transferred to a fresh 96-well plate. Hemoglobin release was quantified by measuring absorbance at 405 nm using a microplate reader (BioTek Epoch). Complete hemolysis (100%) was induced by 0.1% Triton X-100, while PBS served as the negative control (0% hemolysis), and hemolysis percentage was transformed from absorbance by the following formula (I):

$$Hemolysis ( \% ) = ( A b {s}_{Polypeptidesolution} - A b {s}_{PBS} ) / ( A b {s}_{Triton X - 100 ( 0.1 \% )} - {Ab s}_{PBS} \times 100 \% \qquad ( I )$$

Abs_{Polypeptide solution} represent absorbance of the copolypeptide solution, Abs_{PBS} represent absorbance of PBS, and Abs_{Triton X-100 (0.1%)} represent absorbance of 0.1% Triton X-100.

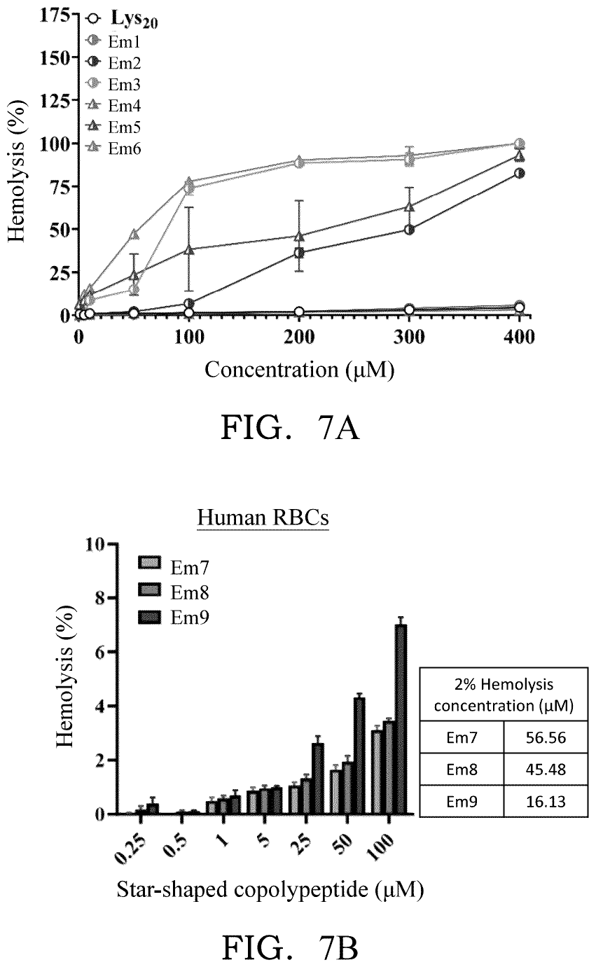

It has been acknowledged that hemolytic activity of Lys₂₀ against RBCs was lower than 5%, and thus Lys₂₀ served as control in this experimental example. As shown in FIG. 7A, compared to Em2 (Lys₂₀-Leu₁₀), Em3 (Lys₂₀-Phe₁₀), Em5 (3s-Lys₁₀-Leu₅) and Em6 (3s-Lys₁₀-Phe₅), Lys₂₀, Em1 (Lys₂₀-Ala10) and Em4 (3s-Lys₁₀-Ala₅) exhibited subtle hemolytic activity. It demonstrated that the copolypeptides tethering hydrophobic segments, including Leun and Phen repeats, resulted in significant hemolytic activity, and such copolypeptides, in lack of biocompatibility, are not conducive to in-body circulatory delivery.

The hydrophobic segment based on Alan are anticipated to provide copolypeptide more biocompatibility when serving as nucleic acid carriers. Notably, as exhibited in FIG. 7B, with increasing number of copolypeptide chains and concentration, hemolytic activities of Em7 (6s-PLL₁₀-PLA₅), Em8 (12s-PLL₁₀-PLA₅) and Em9 (24s-PLL₁₀-PLA₅) were less than 10%, exhibiting excellent biocompatibility. Among all these tests, hemolytic activities of Em7 to Em9 remained less than 5% at 50 μM, and such concentration or a lower one are deemed suitable for the subsequent in vivo or in vitro tests.

### Experimental Example 3.1: Organoid Cytotoxicity Tests I

NIH/3T3 and BEAS-2B cells were cultured in the round bottom 96-well plate at cell density of 2×10⁴ cells/well. After 24 h incubation with copolypeptides from Em1 to Em6, the organoid viability was evaluated using the WST-8 reagent. Cell viability was transformed from absorbance by the following formula (II):

$$Cell viability ( \% ) = ( O {D}_{s a mple} - {OD}_{b l a n k} ) / ( O {D}_{normal} - O {D}_{b l a n k} ) \times 100 \% \qquad ( II )$$

In the formula (II), ODₛₐₘₚₗₑ, OD_{blank} and ODₙₒᵣₘₐₗ represented the absorbance of WST-8 reagent. ODₛₐₘₚₗₑ represented cells treated with copolypeptide in the well. OD_{blank} represented wells with only cell culture medium containing WST-8 reagent. ODₙₒᵣₘₐₗ represented cells without any treatment in the well.

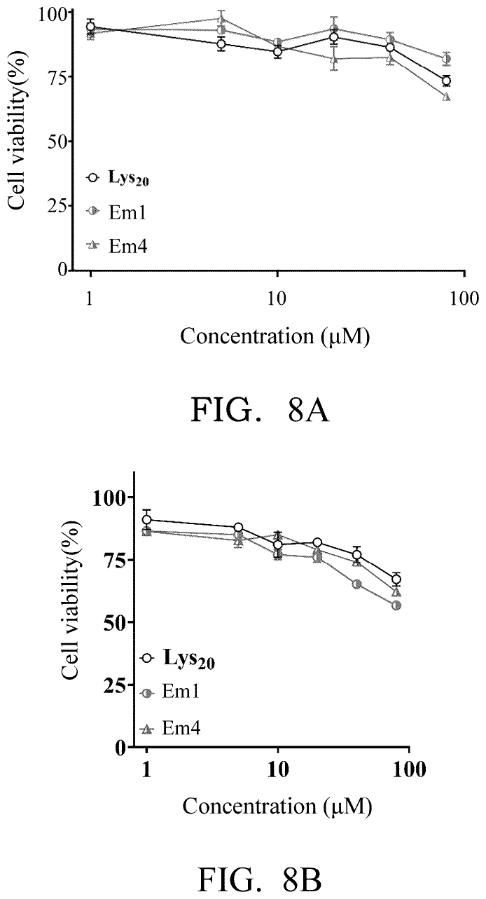

The cell viability tests of NIH/3T3 and BEAS-2B cell spheres after treatment of Lys₂₀, Em1 (Lys₂₀-Ala₁₀) and Em4 (3s-Lys₁₀-Ala₅) were further assessed. As exhibited in FIG. 8A, the cell viability of NIH/3T3 cell organoids was higher than 80% as the organoids treated with the copolypeptides at concentrations lower than 40 μM. In contrast, as exhibited in FIG. 8B, the cell viability of BEAS-2B cell organoids was over 80% as the organoid treated with the copolypeptides at concentrations lower than 20 μM. Overall, copolypeptides of Em1 and Em4 demonstrated excellent biocompatibility.

### Experimental Example 3.2: Organoid Cytotoxicity Tests II

BEAS-2B, HEK-293T, and H1299 cells were seeded in 96-well plates and round-bottom 96-well plates at a cell density of 2×10⁴ cells/well and cultured in 10% CCS/DMEM for 24 hours. Subsequently, serum-free media containing various concentrations of star-shaped copolypeptides of Em7 to Em9 were added to each well. After 20-24 hours of incubation, the medium was replaced with 10% CCK-8 reagent to measure cell viability of each cell line so as to assess cytotoxicity of each copolypeptide. All experiments were performed in quadruplicate.

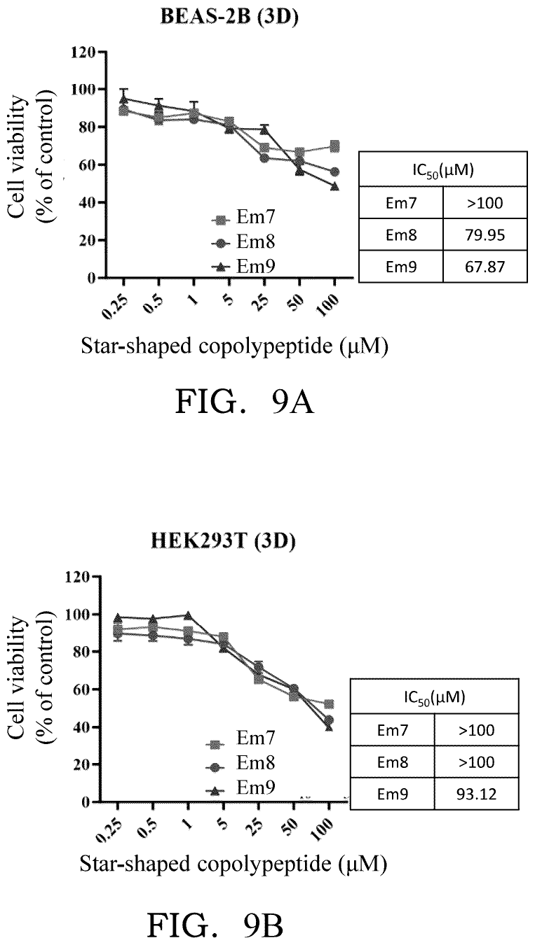

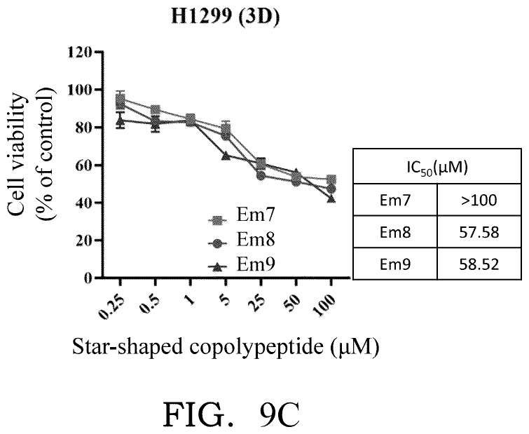

As BEAS-2B, HEK-293T, and H1299 cells grew into cell spheres, and such 3D organoid could mimic in vivo microenvironments. As indicated in FIGS. 9A to 9C, with escalating number of copolypeptide chains (1), IC₅₀ value decreased therewith. Compared to Em7 tethering 6 arms, Em8 with 12 arms and Em9 with 24 arms exhibited higher cytotoxicity across all cell lines tested. In consideration of biocompatibility, Em7 was used in the subsequent transfection efficiency tests.

### Experimental Example 4: In Vitro DNA Transfection

Cells were seeded at a density of 3× 10⁴ cells/well in round-bottom 96-well plates and cultured in complete medium for 18-24 hours to reach 50-70% confluence. Prepared copolypeptide-DNA complexes in Experimental Example 1 with varying N/P ratios were diluted in 2% CCS, and DNA amount was set to be 14 μg DNA/well. Cells were incubated with the copolypeptide-DNA complexes for 24 hours under standard culture conditions. The medium was then replaced with fresh medium containing 10% CCS, and cells were incubated for an additional 24 hours. EGFP expression was quantified by flow cytometry to assess DNA transfection efficiency.

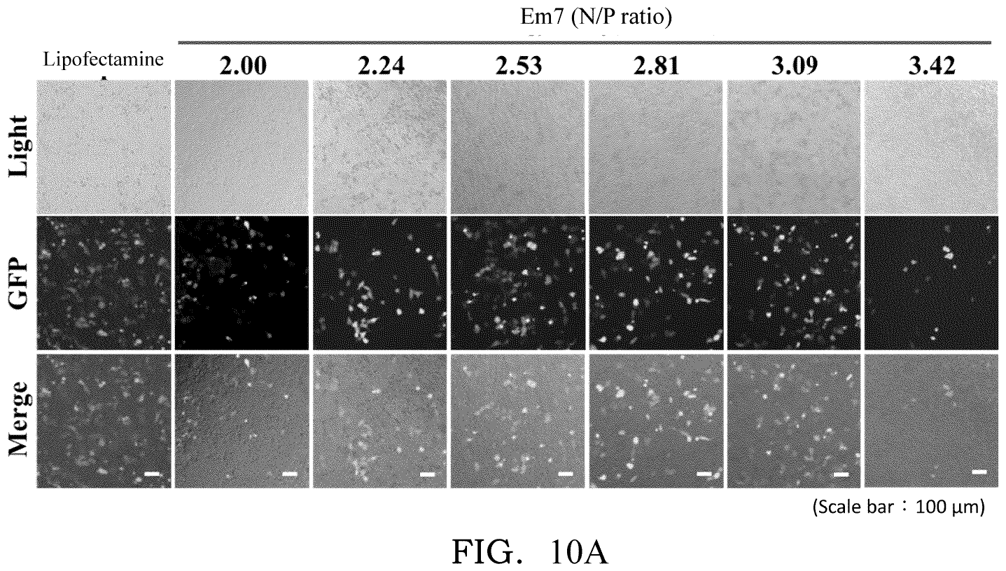

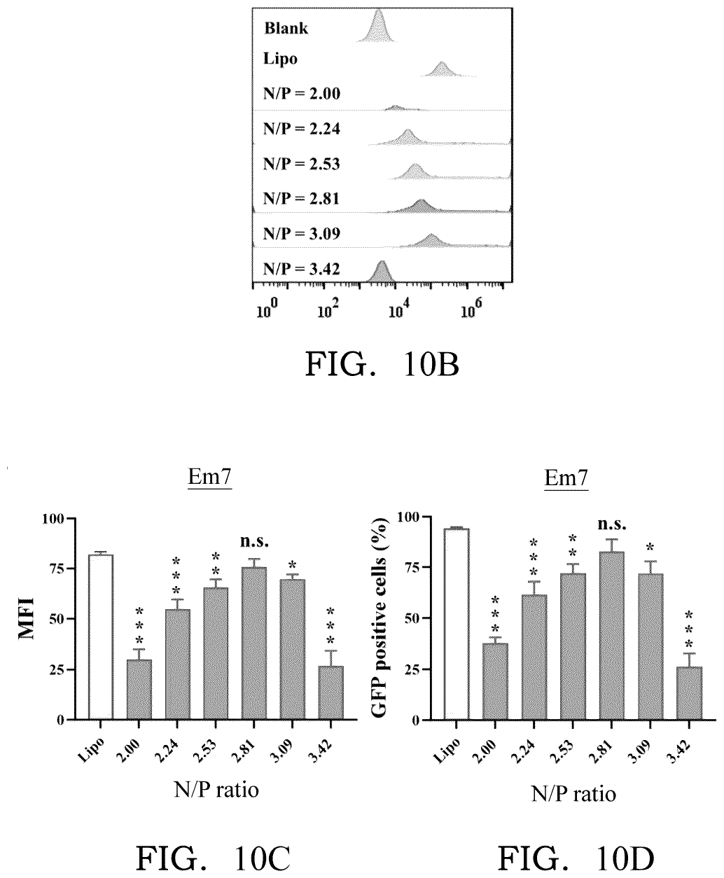

With reference to FIG. 10A, in 2D cell culture models, the star-shaped copolypeptide of Em7 maintained excellent transfection efficiency after 40-hour transfection. In view of FIG. 10B, flow cytometric analysis also verified transfection efficacy of Em7. Further in view of FIG. 10C to 10D, under N/P ratios of 2.53 to 3.09, Em7 achieved fair transfection efficiency at around 65% to 89%, and exhibited the highest transfection efficiency of 89% at an N/P ratio of 2.81. Compared to this, the star-shaped copolypeptides of Em8 and Em9 achieved transfection efficiencies of 14.4% and 11%, respectively, at N/P ratios of approximately 3.99 and 4.98, and their transfection efficiencies were even lower under the same conditions. Em7 demonstrating high and persistent transfection efficiency indicated that copolypeptide-nucleic acid complex (300) not only promoted cells' engulfment of the nucleic acid fragment (200), but also assisted the nucleic acid fragment (200) to escape endosomal degradation. Compared to Em7, with increasing number of arms may result in over-dense hydrophobic peptide segments (12) on the outer surface of the peptide-type nucleic acid carrier (200), which hinders the nucleic acid fragment (200) from contacting with the hydrophobic peptide segment (11) in the inner side of the peptide-type nucleic acid carrier (200) so that delivery efficiency of the carrier is reduced.

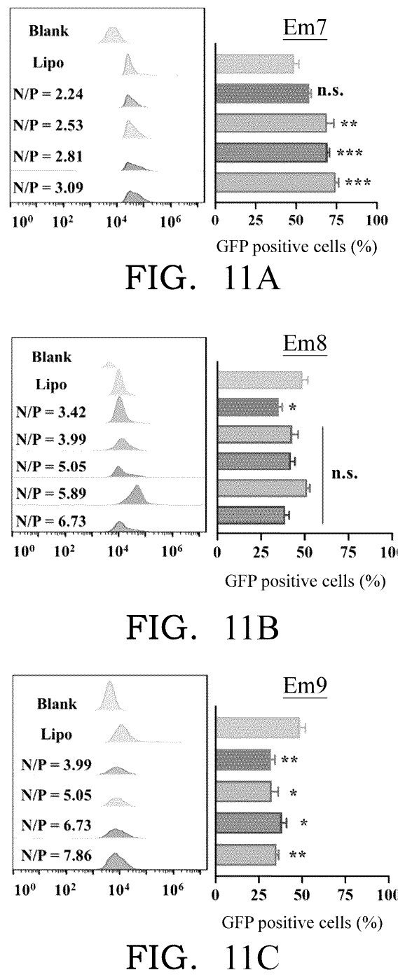

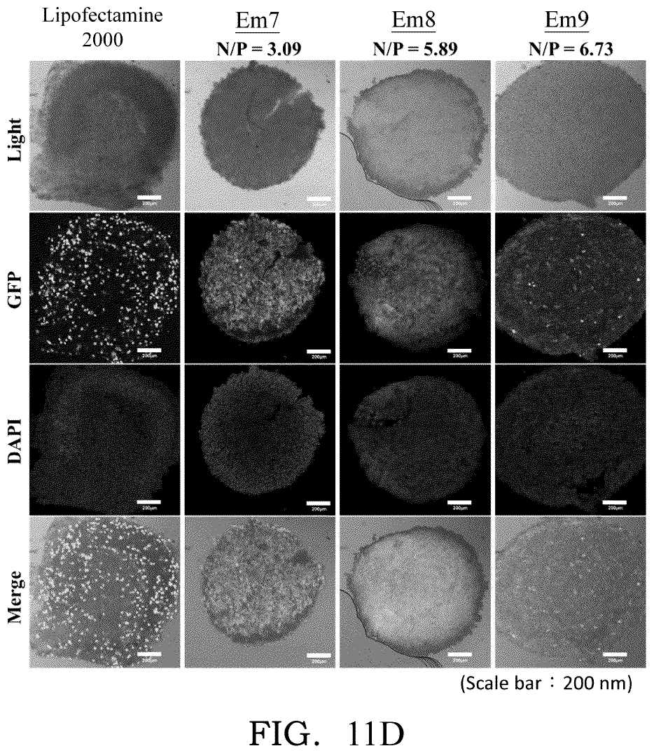

Please refer to FIGS. 11A to 11D, in 3D organoid model, the star-shaped copolypeptides of Em7 to Em9 exhibited transfection efficiencies comparable to Lipofectamine 2000. Among all the tests, as shown in FIG. 11A, Lipofectamine 2000, serving as control, only achieved at 50% transfection efficiency, while Em7 achieved 74% transfection efficiency at N/P ratio of 3.09. Compared to this, as shown in FIGS. 11B to 11C, Em8 and Em9 achieved at 51% and 39%, respectively, under N/P ratios at 5.89 and 6.73. Further in view of FIG. 11D, Z-scan images confirmed deep penetration and efficient transfection of Em7 to Em9 within organoids. Collectively, Em7 demonstrating superior transfection efficiency could serve as a promising transfectant in both 2D and 3D cell culture models.

### Experimental Example 5: In Vitro shRNA Plasmid Transfection and Immunoblotting

MDA-MB-231 breast cancer cells were seeded at a density of 3×10⁴ cells/well in round-bottom 96-well plates and cultured in complete medium for 18-24 hours to reach 50-70% confluence. The cells were then transfected with HSP90 AB1 shRNA plasmid (2.5 nmol, Synbio Technologies) using in Em7. Prepared copolypeptide-shRNA-plasmid complexes with varying N/P ratios were diluted in serum-free medium. Cells were incubated 2.5 nmol shRNA plasmid each well (10 μL/well) for 12 hours under standard conditions. The medium was replaced with fresh medium containing 10% FBS, and the cells were incubated for an additional 36 hours. Transfection efficiency was assessed after 48 hours.

Cells were washed twice with PBS and lysed with RIPA lysis buffer. Immunoblotting was performed using primary antibodies against HSP90 beta (GTX101448, GeneTex), PARP (ab137653, Abcam), GAPDH (6C5, Abcam) and β-actin (A3854, Sigma-Aldrich), followed by incubation with corresponding secondary antibodies. Protein-antibody interactions were detected using an enhanced chemiluminescence (ECL) kit (Millipore Immobilon). Band intensities were quantified using ImageJ software after normalization to β-actin to assess transfection efficiency.

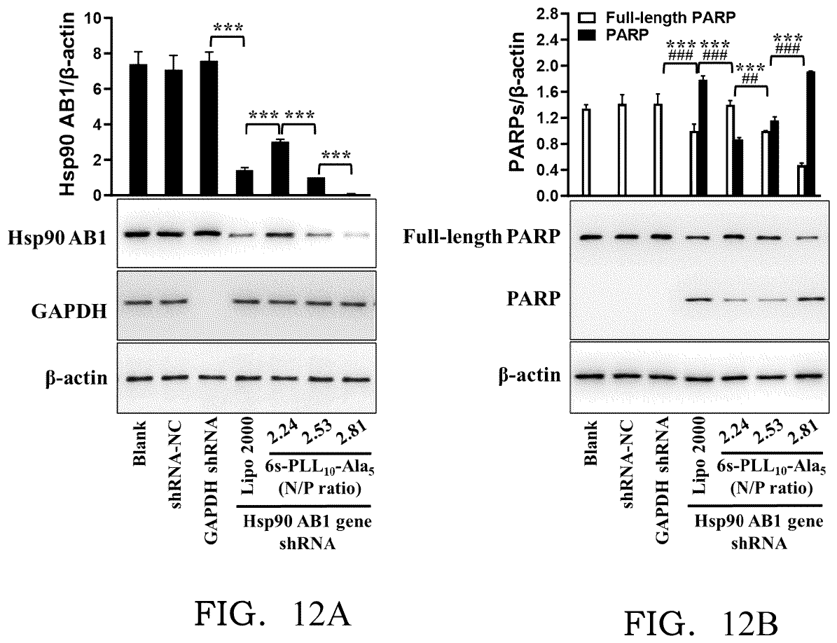

Overexpression of HSP90 AB1 in MDA-MB-231 cells is attributed to aberrant oncogene activation, a hallmark of cellular transformation. As depicted in FIG. 12A, a dose-dependent knockdown of the HSP90 AB gene was achieved using Em7 at varying N/P ratios after 48 hours of transfection. Furthermore, these organoids exhibited induced apoptotic signaling, as indicated by PARP expression after 24 hours. The Experimental Example 6 suggests that targeting HSP90 AB1 with the 6-arm star-shaped copolypeptide (6s-PLL₁₀-PLA₅) carrier prepared in Em7 holds promise as a potential therapeutic strategy for invasive MDA-MB-231 breast cancer. The 6-arm star-shaped copolypeptide in Em7 enables efficient and precise delivery of HSP90 AB1 shRNA plasmids to breast cancer cells, exhibiting high efficiency of HSP90 AB1 gene suppression, and simultaneously activates cell apoptosis, which suggests Em7 is not only capable of delivering plasmid DNA through cell membranes but also ensure integrity of plasmid DNA.

Data in above Experimental Examples are presented as mean #SEM. Statistical analysis was performed using two-way repeated measures ANOVA. Differences between test and control groups were considered significant at *p≤0.05, ** p≤0.01, *** p≤0.001, and **** p≤0.0001.

What is claimed is:

1. A peptide-type nucleic acid carrier, comprising a copolypeptide chain, wherein the copolypeptide chain comprises:
a hydrophilic peptide segment consisting of 5 to 20 substituted or non-substituted amino acids selecting from a group consisting of L-Lysine, L-Arginine, L-Histidine, L-Ornithine and L-Homoarginine; and
a hydrophobic peptide segment, disposed at C-terminus or N-terminus of the hydrophilic peptide segment, consisting of 5 to 20 substituted or non-substituted amino acids selecting from a group consisting of L-Alanine, L-Valine, L-Tryptophan, L-Tyrosine, and L-Methionine.

2. The peptide-type nucleic acid carrier according to claim 1, wherein:
the peptide-type nucleic acid carrier is a branched copolypeptide comprising at least two the copolypeptide chains, wherein N-terminus of any one of the copolypeptide chains links to a side chain of any one of amino acid residues of the hydrophilic peptide segment or the hydrophobic peptide segment of the other of the copolypeptide chains; or
the peptide-type nucleic acid carrier is a star-shaped copolypeptide comprising a core and at least three the copolypeptide chains radially extending from the core, wherein N-terminus or C-terminus of the hydrophilic peptide segment of every one of the copolypeptide chains links to the core.

3. The peptide-type nucleic acid carrier according to claim 1, wherein the star-shaped copolypeptide comprises at least 3 to 24 the copolypeptide chains.

4. The peptide-type nucleic acid carrier according to claim 1, wherein the hydrophilic peptide segment is consisting of substituted or non-substituted amino acids comprising L-Lysine or L-Homoarginine; the hydrophobic peptide segment is consisting of substituted or non-substituted amino acids comprising L-Alanine or L-Valine.

5. The peptide-type nucleic acid carrier according to claim 4, wherein the hydrophilic peptide segment is consisting of substituted or non-substituted L-Lysine; the hydrophobic peptide segment is consisting of substituted or non-substituted L-Alanine.

6. The peptide-type nucleic acid carrier according to claim 1, wherein a ratio of a length of the hydrophilic peptide segment to a length of the hydrophobic peptide segment is (2 to 4): 1.

7. A gene therapeutic composition comprising:
the peptide-type nucleic acid carrier according to claim 1; and
a nucleic acid fragment docking on the peptide-type nucleic acid carrier via electrostatic interaction between a negatively-charged phosphate group and the hydrophilic peptide segment.

8. The gene therapeutic composition according to claim 7, wherein a N/P ratio of the the peptide-type nucleic acid carrier to the nucleic acid fragment is 2.3 to 3.3.

9. The gene therapeutic composition according to claim 7, wherein the nucleic acid fragment encodes a CRISPR, a siRNA, a shRNA, a miRNA, a RNAi, an antisense RNA, a nuclease, or a DNase.

10. The gene therapeutic composition according to claim 7, wherein:
the peptide-type nucleic acid carrier is a branched copolypeptide comprising at least two the copolypeptide chains, wherein N-terminus of any one of the copolypeptide chains links to a side chain of any one of amino acid residues of the hydrophilic peptide segment or the hydrophobic peptide segment of the other of the copolypeptide chains; or
the peptide-type nucleic acid carrier is a star-shaped copolypeptide comprising a core and at least three the copolypeptide chains radially extending from the core, wherein N-terminus or C-terminus of the hydrophilic peptide segment of every one of the copolypeptide chains links to the core.

11. The gene therapeutic composition according to claim 7, wherein the star-shaped copolypeptide comprises at least 3 to 24 the copolypeptide chains.

12. The gene therapeutic composition according to claim 7, wherein the hydrophilic peptide segment is consisting of substituted or non-substituted amino acids comprising L-Lysine or L-Homoarginine; the hydrophobic peptide segment is consisting of substituted or non-substituted amino acids comprising L-Alanine or L-Valine.

13. The gene therapeutic composition according to claim 12, wherein the hydrophilic peptide segment is consisting of substituted or non-substituted L-Lysine; the hydrophobic peptide segment is consisting of substituted or non-substituted L-Alanine.

14. The gene therapeutic composition according to claim 7, wherein a ratio of a length of the hydrophilic peptide segment to a length of the hydrophobic peptide segment is (2 to 4): 1.

15. A method of delivering a nucleic fragment, comprising:
combining the peptide-type nucleic acid carrier according to claim 1 and a nucleic acid fragment to obtain a copolypeptide-nucleic acid complex; and
contacting the copolypeptide-nucleic acid complex to a phospholipid bilayer membrane of a subject in need, wherein:
as the hydrophobic peptide segment and a hydrophilic surface area of the phospholipid bilayer membrane mutually repulse each other, and the hydrophobic peptide segment creates hydrophobic effect with a hydrophobic area on internal side of the phospholipid bilayer, the copolypeptide-nucleic acid complex enters the phospholipid bilayer membrane; the copolypeptide-nucleic acid complex moves along a concentration gradient across two sides of the phospholipid bilayer membrane, thereby making the copolypeptide-nucleic acid complex penetrate the phospholipid bilayer membrane and enter the subject in need.

16. The method according to claim 13, wherein a N/P ratio of the the peptide-type nucleic acid carrier to the nucleic acid fragment is 2.3 to 3.3.

17. The method according to claim 13, wherein the nucleic acid fragment encodes a CRISPR, a siRNA, a shRNA, a miRNA, a RNAi, an antisense RNA, a nuclease, or a DNase.

18. The method according to claim 13, wherein:
the peptide-type nucleic acid carrier is a branched copolypeptide comprising at least two the copolypeptide chains, wherein N-terminus of any one of the copolypeptide chains links to a side chain of any one of amino acid residues of the hydrophilic peptide segment or the hydrophobic peptide segment of the other of the copolypeptide chains; or
the peptide-type nucleic acid carrier is a star-shaped copolypeptide comprising a core and at least three the copolypeptide chains radially extending from the core, wherein N-terminus or C-terminus of the hydrophilic peptide segment of every one of the copolypeptide chains links to the core.

19. The method according to claim 13, wherein the star-shaped copolypeptide comprises at least 3 to 24 the copolypeptide chains; the hydrophilic peptide segment is consisting of substituted or non-substituted amino acids comprising L-Lysine or L-Homoarginine; the hydrophobic peptide segment is consisting of substituted or non-substituted amino acids comprising L-Alanine or L-Valine.

20. The method according to claim 15, wherein a ratio of a length of the hydrophilic peptide segment to a length of the hydrophobic peptide segment is (2 to 4): 1.
